(19) United States
(12) Patent Application Publication
(10) Pub. No.: US 2024/0335093 A1
(43) Pub. Date: Oct. 10, 2024

(54) MEDICAL SUPPORT DEVICE, ENDOSCOPE SYSTEM, MEDICAL SUPPORT METHOD, AND PROGRAM

(71) Applicant: FUJIFILM Corporation, Tokyo (JP)

(72) Inventor: Hiroki WATANABE, Kanagawa (JP)

(73) Assignee: FUJIFILM Corporation, Tokyo (JP)

(21) Appl. No.: 18/608,161

(22) Filed: Mar. 18, 2024

(30) Foreign Application Priority Data
Apr. 10, 2023 (JP) 2023-063565

(51) Int. Cl. A61B 1/00; G06T 7/00

(52) U.S. Cl. CPC A61B 1/000096; A61B 1/000094; A61B 1/00045; G06T 7/0012; G06T 2207/10068; G06T 2207/30096

(57) ABSTRACT

A medical support device includes a processor. An actual size estimation AI is operated to estimate an actual size of a region to be observed which has been recognized by performing a recognition process on a medical image including the region to be observed. The processor is configured to acquire the actual size and to operate the actual size estimation AI according to a recognition result obtained by the recognition process.

### CROSS-REFERENCE TO RELATED APPLICATIONS

This application claims priority under 35 USC 119 from Japanese Patent Application No. 2023-063565 filed on Apr. 10, 2023, the disclosure of which is incorporated by reference herein.

### BACKGROUND

### 1. Technical Field

The technology of the present disclosure relates to a medical support device, an endoscope system, a medical support method, and a program.

### 2. Related Art

JP2020-093076A discloses an image processing device that can detect a boundary of a retinal layer regardless of a disease, a part, or the like. The image processing device disclosed in JP2020-093076A comprises an acquisition unit that acquires a tomographic image of an eye to be examined and a first processing unit that executes a first detection process for detecting at least one of a plurality of retinal layers in the acquired tomographic image using a trained model obtained by learning data indicating at least one of the plurality of retinal layers in the tomographic image of the eye to be examined.

WO2020/008651A discloses an endoscope image processing device comprising an image input unit, a lesion detection unit, and a display control output unit. In the endoscope image processing device described in WO2020/008651A, a plurality of observation images obtained by imaging a subject with an endoscope are sequentially input to the image input unit. The lesion detection unit detects a lesion part, which is an object to be observed with an endoscope, from the observation image. The display control output unit adds a detection result of the lesion part to the observation image and outputs the observation image. The display control output unit comprises a lesion state analysis unit that analyzes a state of the lesion and a display extension time setting unit that sets a display extension time of the detection result of the lesion part according to the state of the lesion part.

In the endoscope image processing device described in WO2020/008651A, the lesion state analysis unit comprises a visibility analysis unit that analyzes visibility of the lesion part. The visibility analysis unit comprises a lesion unit information analysis unit that analyzes the visibility of each lesion part. The lesion unit information analysis unit comprises a lesion size estimation unit that estimates a size of the lesion part. The lesion unit information analysis unit comprises a lesion position analysis unit that analyzes a position of the lesion part in the observation image.

WO2020/165978A discloses an image recording device including an acquisition unit that acquires time-series images of endoscopy, a lesion appearance specification unit that specifies appearance of a lesion in the acquired time-series images, and a recording unit that starts recording of the time-series images from the time when the lesion appearance specification unit specified the appearance of the lesion. In the image recording device disclosed in WO2020/165978A, the lesion appearance specification unit includes a lesion detection unit that detects a lesion on the basis of the acquired time-series images. The lesion appearance specification unit further includes a lesion information calculation unit that calculates information related to the lesion on the basis of the lesion detected by the lesion detection unit. The lesion information calculation unit calculates position information related to the lesion detected by the lesion detection unit. The lesion information calculation unit calculates information related to the size of the lesion detected by the lesion detection unit.

### SUMMARY

An embodiment according to the technology of the present disclosure provides a medical support device, an endoscope system, a medical support method, and a program that can achieve both highly accurate estimation of an actual size of a region to be observed and suppression of an increase in calculation load caused by an operation of an actual size estimation AI.

According to a first aspect of the technology of the present disclosure, there is provided a medical support device comprising a processor. An actual size estimation AI is operated to estimate an actual size of a region to be observed which has been recognized by performing a recognition process on a medical image including the region to be observed. The processor is configured to: acquire the actual size; and operate the actual size estimation AI according to a recognition result obtained by the recognition process.

According to a second aspect of the technology of the present disclosure, in the medical support device according to the first aspect, the recognition result may include geometric characteristics of the region to be observed in the medical image.

According to a third aspect of the technology of the present disclosure, in the medical support device according to the second aspect, the geometric characteristics may include a position and/or an approximate size of the region to be observed in the medical image.

According to a fourth aspect of the technology of the present disclosure, in the medical support device according to the third aspect, the processor may be configured to operate the actual size estimation AI according to an amount of change in the position over time and/or an amount of change in the approximate size over time.

According to a fifth aspect of the technology of the present disclosure, in the medical support device according to the fourth aspect, the processor may be configured to operate the actual size estimation AI in a case where the amount of change in the position over time is equal to or less than a first threshold value and/or in a case where the amount of change in the approximate size over time is equal to or less than a second threshold value.

According to a sixth aspect of the technology of the present disclosure, in the medical support device according to the fourth aspect, the processor may be configured to operate the actual size estimation AI in a case where a first value obtained by smoothing the amount of change in the position over time is equal to or less than a first threshold value and/or in a case where a second value obtained by smoothing the amount of change in the approximate size over time is equal to or less than a second threshold value.

According to a seventh aspect of the technology of the present disclosure, in the medical support device according to the sixth aspect, the first value may be a moving average value of the amount of change in the position over time.

According to an eighth aspect of the technology of the present disclosure, in the medical support device according to the sixth aspect or the seventh aspect, the second value may be a moving average value of the amount of change in the approximate size over time.

According to a ninth aspect of the technology of the present disclosure, in the medical support device according to any one of the third to eighth aspects, the recognition process may be a process that uses an object recognition AI using a bounding box method, and the approximate size may be a bounding box size that is applied to the region to be observed by the object recognition AI.

According to a tenth aspect of the technology of the present disclosure, in the medical support device according to any one of the third to ninth aspects, the geometric characteristics may include the position, and the processor may be configured to operate the actual size estimation AI on a condition that the position is present in a closed region surrounding a portion of the medical image.

According to an eleventh aspect of the technology of the present disclosure, in the medical support device according to the tenth aspect, the closed region may be set in a central portion of the medical image.

According to a twelfth aspect of the technology of the present disclosure, in the medical support device according to the tenth aspect or the eleventh aspect, the closed region may be set in the medical image in response to a given instruction.

According to a thirteenth aspect of the technology of the present disclosure, in the medical support device according to any one of the first to twelfth aspects, a plurality of the regions to be observed may be included in the medical image, and the recognition process may be a process of recognizing each of the plurality of regions to be observed which are included in the medical image. The recognition result may be obtained for each of the plurality of regions to be observed, and the actual size estimation AI may be operated for each of the plurality of regions to be observed which have been recognized by performing the recognition process on the medical image. The processor may be configured to operate the actual size estimation AI for each of the plurality of regions to be observed according to the recognition result for each of the plurality of regions to be observed.

According to a fourteenth aspect of the technology of the present disclosure, in the medical support device according to any one of the first to thirteenth aspects, in a case where there is a time period in which the recognition result is not obtained by the recognition process, the recognition result in the time period may be interpolated on the basis of the recognition result obtained by the recognition process before and/or after the time period.

According to a fifteenth aspect of the technology of the present disclosure, in the medical support device according to any one of the first to fourteenth aspects, the processor may be configured to perform the recognition process on the medical image.

According to a sixteenth aspect of the technology of the present disclosure, in the medical support device according to any one of the first to fifteenth aspects, the processor may be configured to operate the actual size estimation AI to estimate the actual size.

According to a seventeenth aspect of the technology of the present disclosure, in the medical support device according to any one of the first to sixteenth aspects, the processor may be configured to output the actual size.

According to an eighteenth aspect of the technology of the present disclosure, in the medical support device according to the seventeenth aspect, the output of the actual size may be implemented by displaying the actual size on a screen.

According to a nineteenth aspect of the technology of the present disclosure, in the medical support device according to any one of the first to eighteenth aspects, the processor may be configured to output operating state information that is capable of specifying an operating state of the actual size estimation AI.

According to a twentieth aspect of the technology of the present disclosure, in the medical support device according to the nineteenth aspect, the output of the operating state information may be implemented by displaying the operating state information on a screen.

According to a twenty-first aspect of the technology of the present disclosure, in the medical support device according to any one of the first to twentieth aspects, the medical image may be an endoscope image captured by an endoscope.

According to a twenty-second aspect of the technology of the present disclosure, in the medical support device according to any one of the first to twenty-first aspects, the region to be observed may be a lesion.

According to a twenty-third aspect of the technology of the present disclosure, there is provided an endoscope system comprising: the medical support device according to any one of the first to twenty-second aspects; and an endoscope that images a region to be observed.

According to a twenty-fourth aspect of the technology of the present disclosure, there is provided a medical support method comprising: operating an actual size estimation AI to estimate an actual size of a region to be observed which has been recognized by performing a recognition process on a medical image including the region to be observed; acquiring the actual size; and operating the actual size estimation AI according to a recognition result obtained by the recognition process.

According to a twenty-fifth aspect of the technology of the present disclosure, the medical support method according to the twenty-fourth aspect may further comprise using an endoscope that images the region to be observed.

According to a twenty-sixth aspect of the technology of the present disclosure, there is provided a program causing a computer to execute a medical support process comprising: operating an actual size estimation AI to estimate an actual size of a region to be observed which has been recognized by performing a recognition process on a medical image including the region to be observed; acquiring the actual size; and operating the actual size estimation AI according to a recognition result obtained by the recognition process.

### BRIEF DESCRIPTION OF THE DRAWINGS

Exemplary embodiments of the technology of the disclosure will be described in detail based on the following figures, wherein:

FIG. 1 is a conceptual diagram illustrating an example of an aspect in which an endoscope system is used;

FIG. 2 is a conceptual diagram illustrating an example of an overall configuration of the endoscope system;

FIG. 3 is a block diagram illustrating an example of a hardware configuration of an electrical system of the endoscope system;

FIG. 4 is a block diagram illustrating an example of functions of main units of a processor included in a medical support device according to an embodiment and an example of information stored in an NVM;

FIG. 5 is a conceptual diagram illustrating an example of content of processes of a recognition unit and a control unit;

FIG. 6 is a conceptual diagram illustrating an example of content of a process of an acquisition unit;

FIG. 7 is a conceptual diagram illustrating an example of the content of the process of the control unit;

FIG. 8 is a conceptual diagram illustrating an example of an aspect in which various types of information are displayed in a first display region and a second display region;

FIG. 9 is a flowchart illustrating an example of a flow of a medical support process;

FIG. 10 is a conceptual diagram illustrating a first modification example of the content of the process of the control unit;

FIG. 11 is a conceptual diagram illustrating a modification example of the content of the process of the recognition unit;

FIG. 12 is a conceptual diagram illustrating a second modification example of the content of the process of the control unit;

FIG. 13 is a conceptual diagram illustrating a third modification example of the content of the process of the control unit;

FIG. 14 is a conceptual diagram illustrating a fourth modification example of the content of the process of the control unit;

FIG. 15 is a conceptual diagram illustrating a fifth modification example of the content of the process of the control unit;

FIG. 16 is a conceptual diagram illustrating an example of content of a process performed by the processor in a case where a plurality of lesions are included in a frame;

FIG. 17 is a conceptual diagram illustrating an example of an output destination of various types of information; and

FIG. 18 is a conceptual diagram illustrating an example of a series of processes in which the processor of the endoscope system gives a process execution request to an external device via a network, the external device executes the process in response to the process execution request, and the processor of the endoscope system receives a result of the process from the external device.

### DETAILED DESCRIPTION

Hereinafter, examples of embodiments of a medical support device, an endoscope system, a medical support method, and a program according to the technology of the present disclosure will be described with reference to the accompanying drawings.

First, the wording used in the following description will be described.

CPU refers to an abbreviation of “Central Processing Unit”. GPU is an abbreviation of “Graphics Processing Unit”. RAM is an abbreviation of “Random Access Memory”. NVM is an abbreviation of “Non-Volatile Memory”. EEPROM is an abbreviation of “Electrically Erasable Programmable Read-Only Memory”. ASIC is an abbreviation of “Application Specific Integrated Circuit”. PLD is an abbreviation of “Programmable Logic Device”. FPGA is an abbreviation of “Field-Programmable Gate Array”. SoC is an abbreviation of “System-on-a-Chip”. SSD is an abbreviation of “Solid State Drive”. USB is an abbreviation of “Universal Serial Bus”. HDD is an abbreviation of “Hard Disk Drive”. EL is an abbreviation of “Electro-Luminescence”. CMOS is an abbreviation of “Complementary Metal Oxide Semiconductor”. CCD is an abbreviation of “Charge Coupled Device”. AI is an abbreviation of “Artificial Intelligence”. BLI is an abbreviation of “Blue Light Imaging”. LCI is an abbreviation of “Linked Color Imaging”. I/F is an abbreviation of “Interface”. SSL stands for “Sessile Serrated Lesion”. LAN is an abbreviation of “Local Area Network”. WAN is an abbreviation of “Wide Area Network”.

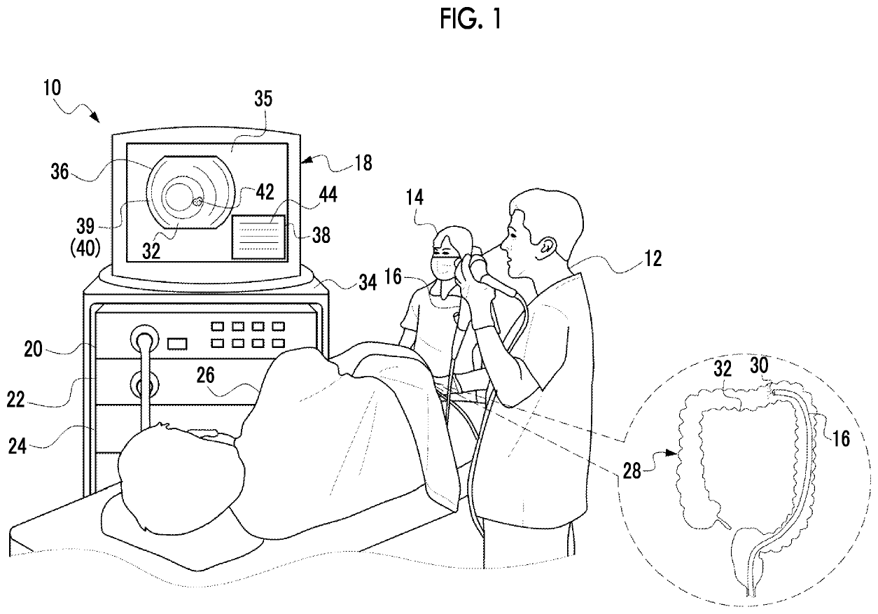

For example, as illustrated in FIG. 1, an endoscope system 10 is used by a doctor 12 in endoscopy or the like. The endoscopy is assisted by a staff member such as a nurse 14. In this embodiment, the endoscope system 10 is an example of an “endoscope system” according to the technology of the present disclosure.

The endoscope system 10 is connected to a communication device (not illustrated) to communicate therewith, and information obtained by the endoscope system 10 is transmitted to the communication device. An example of the communication device is a server and/or a client terminal (for example, a personal computer and/or a tablet terminal) that manages various types of information such as electronic medical records. The communication device receives the information transmitted from the endoscope system 10 and executes a process using the received information (for example, a process of storing the information in the electronic medical record or the like).

The endoscope system 10 comprises an endoscope 16, a display device 18, a light source device 20, a control device 22, and a medical support device 24. In this embodiment, the endoscope 16 is an example of an “endoscope” according to the technology of the present disclosure.

The endoscope system 10 is a modality for performing a medical examination on a large intestine 28 included in a body of a subject 26 (for example, a patient) using the endoscope 16. In this embodiment, the large intestine 28 is an object to be observed by the doctor 12.

The endoscope 16 is used by the doctor 12 and is inserted into a body cavity of the subject 26. In this embodiment, the endoscope 16 is inserted into the large intestine 28 of the subject 26. In the endoscope system 10, the endoscope 16 inserted into the large intestine 28 of the subject 26 is used to image the inside of the large intestine 28 of the subject 26, and various types of medical treatments are performed on the large intestine 28 as necessary.

The endoscope system 10 images the inside of the large intestine 28 of the subject 26 to acquire an image showing an aspect of the inside of the large intestine 28 and outputs the image. In this embodiment, the endoscope system 10 is an endoscope apparatus having an optical imaging function of irradiating the inside of the large intestine 28 with light 30 and capturing reflected light from an intestinal wall 32 of the large intestine 28.

In addition, here, endoscopy for the large intestine 28 is given as an example. However, this is only an example, and the technology of the present disclosure is established even in endoscopy for a hollow organ such as an esophagus, a stomach, a duodenum, or a trachea.

The light source device 20, the control device 22, and the medical support device 24 are installed in a wagon 34. A plurality of tables are provided in the wagon 34 along a vertical direction, and the medical support device 24, the control device 22, and the light source device 20 are installed from a lower table to an upper table. In addition, the display device 18 is installed on the uppermost table in the wagon 34.

The control device 22 controls the entire endoscope system 10. The medical support device 24 performs various types of image processing on the image obtained by imaging the intestinal wall 32 with the endoscope 16 under the control of the control device 22.

The display device 18 displays various types of information including the image. An example of the display device 18 is a liquid crystal display or an EL display. In addition, a tablet terminal with a display may be used instead of the display device 18 or together with the display device 18.

A screen 35 is displayed on the display device 18. The screen 35 includes a plurality of display regions. The plurality of display regions are disposed side by side in the screen 35. In the example illustrated in FIG. 1, a first display region 36 and a second display region 38 are illustrated as an example of the plurality of display regions. A size of the first display region 36 is larger than a size of the second display region 38. The first display region 36 is used as a main display region, and the second display region 38 is used as a sub-display region. Further, a size relationship between the first display region 36 and the second display region 38 is not limited thereto and may be any size relationship that falls within the screen 35. In this embodiment, the screen 35 is an example of a “screen” according to the technology of the present disclosure.

An endoscope video image 39 is displayed in the first display region 36. The endoscope video image 39 is a video image acquired by imaging the intestinal wall 32 with the endoscope 16 in the large intestine 28 of the subject 26. In the example illustrated in FIG. 1, a video image including the intestinal wall 32 is given as an example of the endoscope video image 39.

The intestinal wall 32 included in the endoscope video image 39 includes a lesion 42 (for example, one lesion 42 in the example illustrated in FIG. 1) as a region of interest (that is, a region to be observed) at which the doctor 12 gazes, and the doctor 12 can visually recognize the aspect of the intestinal wall 32 including the lesion 42 through the endoscope video image 39. In this embodiment, the lesion 42 is an example of a “region to be observed” and a “lesion” according to the technology of the present disclosure.

There are various types of lesions 42, and examples of the type of the lesion 42 include a neoplastic polyp and a non-neoplastic polyp. An example of the type of neoplastic polyp is an adenoma polyp (for example, an SSL). Examples of the type of non-neoplastic polyp include a hamartomatous polyp, a hyperplastic polyp, and an inflammatory polyp. In addition, the type given as an example here is a type that is assumed in advance as the type of the lesion 42 in a case where endoscopy is performed on the large intestine 28. In a case where the organ to be subjected to endoscopy is different, the type of lesion is also different.

In this embodiment, for convenience of description, a form in which one lesion 42 is included in the endoscope video image 39 is given as an example. However, the technology of the present disclosure is not limited thereto. The technology of the present disclosure is established even in a case where a plurality of lesions 42 are included in the endoscope video image 39.

In this embodiment, the lesion 42 is given as an example. However, this is only an example. The region of interest (that is, the region to be observed) at which the doctor 12 gazes may be an organ (for example, a duodenal papilla), a marked region, an artificial treatment tool (for example, an artificial clip), a treated region (for example, a region in which a trace of removal of a polyp or the like remains), or the like.

The image displayed in the first display region 36 is one frame 40 that is included in a video image configured to include a plurality of frames 40 arranged in time series. That is, the plurality of frames 40 arranged in time series are displayed in the first display region 36 at a predetermined frame rate (for example, several tens of frames/second). In this embodiment, the frame 40 is an example of a “medical image” and an “endoscope image” according to the technology of the present disclosure.

An example of the video image displayed in the first display region 36 is a video image in a live view mode. The live view mode is only an example, and the video image may be a video image, such as a video image in a post view mode, that is temporarily stored in a memory or the like and then displayed. In addition, each frame included in a recording video image stored in the memory or the like may be reproduced and displayed as the endoscope video image 39 on the screen 35 (for example, in the first display region 36).

In the screen 35, the second display region 38 is adjacent to the first display region 36 and is displayed on the lower right side of the screen 35 in a front view. The second display region 38 may be displayed at any position in the screen 35 of the display device 18 and is preferably displayed at a position that can be contrasted with the endoscope video image 39.

Medical information 44, which is information related to the medical treatment, is displayed in the second display region 38. An example of the medical information 44 is information that assists medical determination or the like by the doctor 12. Examples of the information that assists the medical determination or the like by the doctor 12 include various types of information related to the subject 26 into which the endoscope 16 is inserted and/or various types of information obtained by performing a process using AI on the endoscope video image 39.

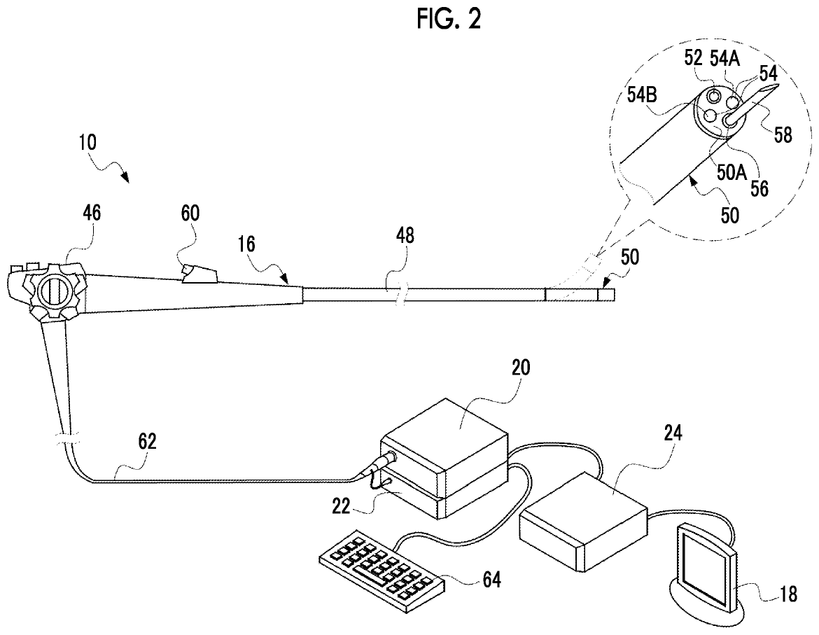

For example, as illustrated in FIG. 2, the endoscope 16 comprises an operation unit 46 and an insertion portion 48. The insertion portion 48 is partially curved by the operation of the operation unit 46. The insertion portion 48 is inserted into the large intestine 28 while being curved according to the shape of the large intestine 28 (see FIG. 1) according to the operation of the operation unit 46 by the doctor 12 (see FIG. 1).

A camera 52, an illumination device 54, and a treatment tool opening 56 are provided in a distal end part 50 of the insertion portion 48. The camera 52 and the illumination device 54 are provided on a distal end surface 50A of the distal end part 50. In addition, here, the form in which the camera 52 and the illumination device 54 are provided on the distal end surface 50A of the distal end part 50 is given as an example. However, this is only an example. The camera 52 and the illumination device 54 may be provided on a side surface of the distal end part 50 such that the endoscope 16 is configured as a side-viewing endoscope.

The camera 52 is inserted into the body cavity of the subject 26 and images the region to be observed. In this embodiment, the camera 52 images the inside of the body of the subject 26 (for example, the inside of the large intestine 28) to acquire the endoscope video image 39. An example of the camera 52 is a CMOS camera. However, this is only an example, and the camera 52 may be other types of cameras such as CCD cameras.

The illumination device 54 has illumination windows 54A and 54B. The illumination device 54 emits the light 30 (see FIG. 1) through the illumination windows 54A and 54B. Examples of the type of the light 30 emitted from the illumination device 54 include visible light (for example, white light) and invisible light (for example, near-infrared light). In addition, the illumination device 54 emits special light through the illumination windows 54A and 54B. Examples of the special light include light for BLI and/or light for LCI. The camera 52 images the inside of the large intestine 28 using an optical method in a state in which the illumination device 54 irradiates the inside of the large intestine 28 with the light 30.

The treatment tool opening 56 is an opening through which a treatment tool 58 protrudes from the distal end part 50. Further, the treatment tool opening 56 is also used as a suction port for sucking blood, body waste, and the like and a delivery port for sending out a fluid.

A treatment tool insertion opening 60 is formed in the operation unit 46, and the treatment tool 58 is inserted into the insertion portion 48 through the treatment tool insertion opening 60. The treatment tool 58 passes through the insertion portion 48 and protrudes from the treatment tool opening 56 to the outside. In the example illustrated in FIG. 2, an aspect in which, as the treatment tool 58, a puncture needle protrudes from the treatment tool opening 56 is illustrated. Here, the puncture needle is given as an example of the treatment tool 58. However, this is only an example. The treatment tool 58 may be grasping forceps, a papillotomy knife, a snare, a catheter, a guide wire, a cannula, and/or a puncture needle with a guide sheath.

The endoscope 16 is connected to the light source device 20 and the control device 22 through a universal cord 62. The medical support device 24 and a receiving device 64 are connected to the control device 22. In addition, the display device 18 is connected to the medical support device 24. That is, the control device 22 is connected to the display device 18 through the medical support device 24.

In addition, here, the medical support device 24 is given as an example of an external device for expanding the functions of the control device 22. Therefore, a form in which the control device 22 and the display device 18 are indirectly connected to each other through the medical support device 24 is given as an example. However, this is only an example. For example, the display device 18 may be directly connected to the control device 22. In this case, for example, the functions of the medical support device 24 may be provided in the control device 22, or the control device 22 may be provided with a function of directing a server (not illustrated) to execute the same process as the process (for example, a medical support process which will be described below) performed by the medical support device 24, receiving a result of the process by the server, and using the result.

The receiving device 64 receives an instruction from the doctor 12 and outputs the received instruction as an electric signal to the control device 22. Examples of the receiving device 64 include a keyboard, a mouse, a touch panel, a foot switch, a microphone, and/or a remote control device.

The control device 22 controls the light source device 20, transmits and receives various signals to and from the camera 52, or transmits and receives various signals to and from the medical support device 24.

The light source device 20 emits light under the control of the control device 22 and supplies the light to the illumination device 54. A light guide is provided in the illumination device 54, and the light supplied from the light source device 20 is emitted from the illumination windows 54A and 54B via the light guide. The control device 22 directs the camera 52 to perform imaging, acquires the endoscope video image 39 (see FIG. 1) from the camera 52, and outputs the endoscope video image 39 to a predetermined output destination (for example, the medical support device 24).

The medical support device 24 performs various types of image processing on the endoscope video image 39 input from the control device 22 to support a medical treatment (here, for example, endoscopy). The medical support device 24 outputs the endoscope video image 39 subjected to various types of image processing to a predetermined output destination (for example, the display device 18).

In addition, here, the form in which the endoscope video image 39 output from the control device 22 is output to the display device 18 through the medical support device 24 has been described as an example. However, this is only an example. For example, the control device 22 and the display device 18 may be connected to each other, and the endoscope video image 39 subjected to the image processing by the medical support device 24 may be displayed on the display device 18 through the control device 22.

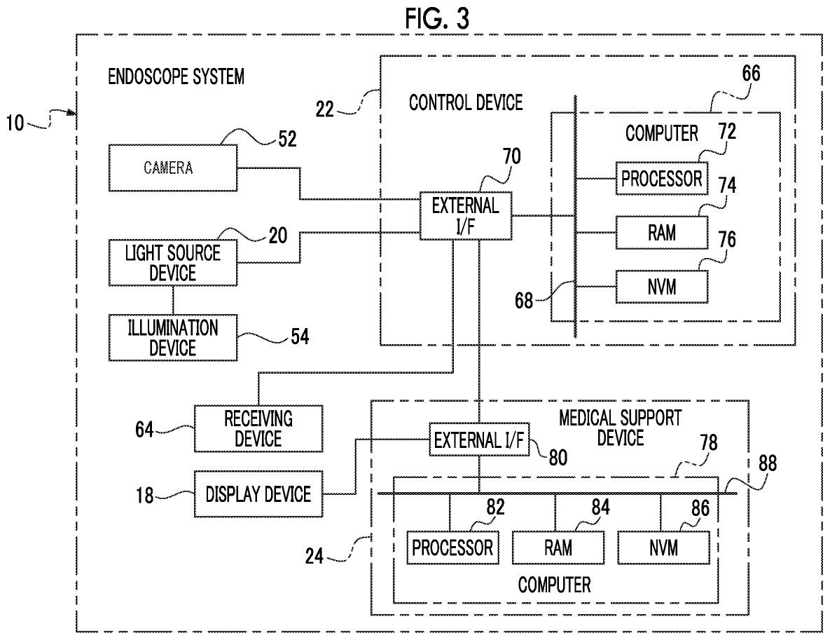

For example, as illustrated in FIG. 3, the control device 22 comprises a computer 66, a bus 68, and an external I/F 70. The computer 66 comprises a processor 72, a RAM 74, and an NVM 76. The processor 72, the RAM 74, the NVM 76, and the external I/F 70 are connected to the bus 68.

For example, the processor 72 includes at least one CPU and at least one GPU and controls the entire control device 22. The GPU operates under the control of the CPU and is in charge of, for example, executing various processes of a graphic system and performing calculation using a neural network. In addition, the processor 72 may be one or more CPUs with which the functions of the GPU have been integrated or may be one or more CPUs with which the functions of the GPU have not been integrated. Further, in the example illustrated in FIG. 3, an aspect in which one processor 72 is mounted on the computer 66 is illustrated. However, this is only an example, and a plurality of processors 72 may be mounted on the computer 66.

The RAM 74 is a memory that temporarily stores information and is used as a work memory by the processor 72. The NVM 76 is a non-volatile storage device that stores, for example, various programs and various parameters. An example of the NVM 76 is a flash memory (for example, an EEPROM and/or an SSD). In addition, the flash memory is only an example, and the NVM 76 may be other non-volatile storage devices, such as HDDs, or a combination of two or more types of non-volatile storage devices.

The external I/F 70 transmits and receives various types of information between one or more devices (hereinafter, also referred to as “first external devices”) outside the control device 22 and the processor 72. A USB interface is given as an example of the external I/F 70.

As one of the first external devices, the camera 52 is connected to the external I/F 70, and the external I/F 70 transmits and receives various types of information between the camera 52 and the processor 72. The processor 72 controls the camera 52 via the external I/F 70. In addition, the processor 72 acquires the endoscope video image 39 (see FIG. 1) obtained by imaging the inside of the large intestine 28 (see FIG. 1) with the camera 52 via the external I/F 70.

As one of the first external devices, the light source device 20 is connected to the external I/F 70, and the external I/F 70 transmits and receives various types of information between the light source device 20 and the processor 72. The light source device 20 supplies light to the illumination device 54 under the control of the processor 72. The illumination device 54 performs irradiation with the light supplied from the light source device 20.

As one of the first external devices, the receiving device 64 is connected to the external I/F 70. The processor 72 acquires the instruction received by the receiving device 64 via the external I/F 70 and executes a process corresponding to the acquired instruction.

The medical support device 24 comprises a computer 78 and an external I/F 80. The computer 78 comprises a processor 82, a RAM 84, and an NVM 86. The processor 82, the RAM 84, the NVM 86, and the external I/F 80 are connected to a bus 88. In this embodiment, the medical support device 24 is an example of a “medical support device” according to the technology of the present disclosure, the computer 78 is an example of a “computer” according to the technology of the present disclosure, and the processor 82 is an example of a “processor” according to the technology of the present disclosure.

In addition, since a hardware configuration of the computer 78 (that is, the processor 82, the RAM 84, and the NVM 86) is basically the same as a hardware configuration of the computer 66, the description of the hardware configuration of the computer 78 will not be repeated here.

The external I/F 80 transmits and receives various types of information between one or more devices (hereinafter, also referred to as “second external devices”) outside the medical support device 24 and the processor 82. A USB interface is given as an example of the external I/F 80.

As one of the second external devices, the control device 22 is connected to the external I/F 80. In the example illustrated in FIG. 3, the external I/F 70 of the control device 22 is connected to the external I/F 80. The external I/F 80 transmits and receives various types of information between the processor 82 of the medical support device 24 and the processor 72 of the control device 22. For example, the processor 82 acquires the endoscope video image 39 (see FIG. 1) from the processor 72 of the control device 22 via the external I/Fs 70 and 80 and performs various types of image processing on the acquired endoscope video image 39.

As one of the second external devices, the display device 18 is connected to the external I/F 80. The processor 82 controls the display device 18 via the external I/F 80 such that various types of information (for example, the endoscope video image 39 subjected to various types of image processing) are displayed on the display device 18.

Meanwhile, in endoscopy, the doctor 12 determines whether or not a medical treatment is necessary for the lesion 42 included in the endoscope video image 39 while checking the endoscope video image 39 through the display device 18 and performs a medical treatment on the lesion 42 as necessary. An actual size, which is the size of the lesion 42 in a real space, is an important determination element in the determination of whether or not a medical treatment is necessary.

In recent years, with the development of machine learning, it has been possible to detect and discriminate the lesion 42 on the basis of the endoscope video image 39 using an AI method. In addition, an attempt has been made to estimate the actual size of the lesion 42 on the basis of the endoscope video image 39 using the AI method. In this case, AI that can estimate the actual size of the lesion 42 is operated to estimate the actual size of the lesion 42, and the estimation result is presented to the doctor 12. As described above, the presentation of the actual size estimated by AI to the doctor 12 is very useful for the doctor 12 to perform a medical treatment on the lesion.

However, there is a concern that a calculation load will increase due to the operation of AI. In addition, in the estimation of the actual size of the lesion 42 by AI on the basis of the endoscope video image 39, in a case where the appearance of the lesion 42 in the endoscope video image 39 changes due to, for example, body movement and/or shake of the camera 52, there is a concern that the actual size estimated by AI will not be stable and an incorrect actual size will be presented to the doctor 12.

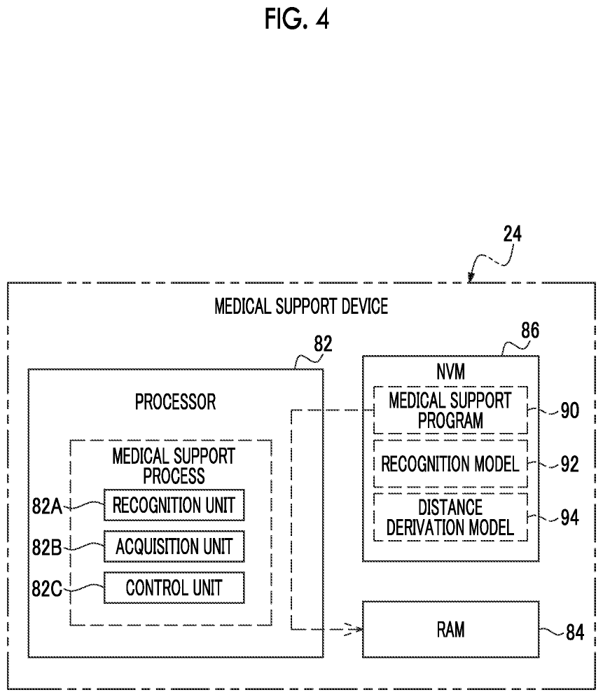

Therefore, in view of these circumstances, in this embodiment, for example, as illustrated in FIG. 4, the processor 82 of the medical support device 24 performs the medical support process.

A medical support program 90 is stored in the NVM 86. The medical support program 90 is an example of a “program” according to the technology of the present disclosure. The processor 82 reads out the medical support program 90 from the NVM 86 and executes the read medical support program 90 on the RAM 84 to perform the medical support process. The processor 82 operates as a recognition unit 82A, an acquisition unit 82B, and a control unit 82C according to the medical support program 90 executed on the RAM 84 to implement the medical support process.

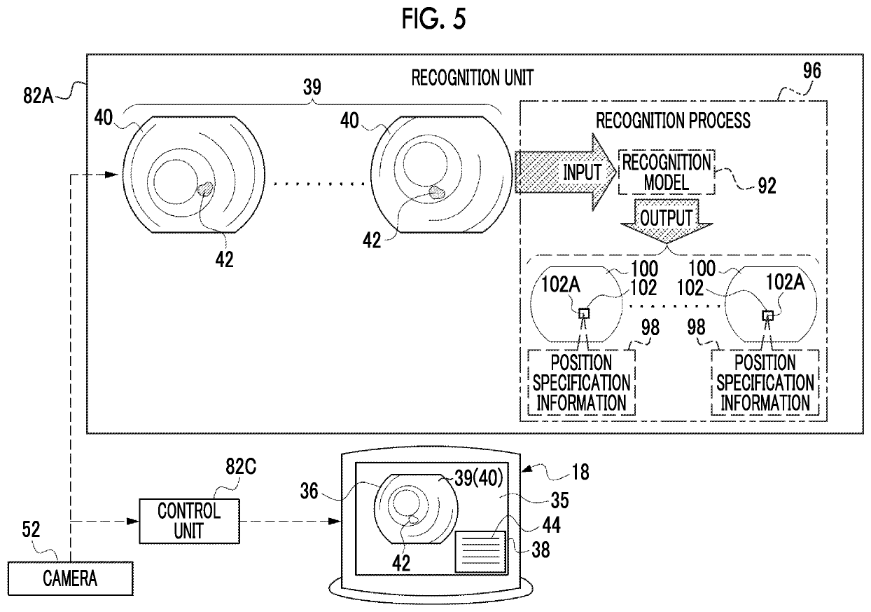

A recognition model 92 and a distance derivation model 94 are stored in the NVM 86. The recognition model 92 is used by the recognition unit 82A, and the distance derivation model 94 is used by the acquisition unit 82B, which will be described in detail below. For example, as illustrated in FIG. 5, the recognition unit 82A and the control unit 82C acquire each of the plurality of frames 40, which are arranged in time series in the endoscope video image 39 captured by the camera 52 according to an imaging frame rate (for example, several tens of frames/second), from the camera 52 frame by frame in time series.

The control unit 82C outputs the endoscope video image 39 to the display device 18. For example, the control unit 82C displays the endoscope video image 39 as a live view image in the first display region 36. That is, each time the control unit 82C acquires the frame 40 from the camera 52, the control unit 82C sequentially displays the acquired frame 40 in the first display region 36 according to a display frame rate (for example, several tens of frames/second). In addition, the control unit 82C displays the medical information 44 in the second display region 38. Further, for example, the control unit 82C updates the display content (for example, the medical information 44) of the second display region 38 according to the display content of the first display region 36.

The recognition unit 82A recognizes the lesion 42 in the endoscope video image 39, using the endoscope video image 39 acquired from the camera 52. That is, the recognition unit 82A sequentially performs a recognition process 96 on each of the plurality of frames 40 arranged in time series in the endoscope video image 39 acquired from the camera 52 to recognize the lesion 42 included in the frame 40. For example, the recognition unit 82A recognizes the geometric characteristics (for example, the position and shape) of the lesion 42, the kind of the lesion 42, and the type of the lesion 42 (for example, a pedunculated type, a semipedunculated type, a sessile type, a superficial elevated type, a superficial flat type, and a superficial depressed type).

The recognition process 96 is performed on the acquired frame 40 each time the recognition unit 82A acquires the frame 40. The recognition process 96 is a process of recognizing the lesion 42 with a method using AI. In this embodiment, for example, an object recognition AI using a bounding box method is used as the recognition process 96.

Here, a process using the recognition model 92 is performed as the recognition process 96. The recognition model 92 is a trained model for object recognition in the bounding box method using AI.

The recognition model 92 is optimized by performing machine learning on the neural network using first training data. The first training data is a data set including a plurality of data items (that is, data corresponding to a plurality of frames) in which first example data and first correct answer data have been associated with each other.

The first example data is an image corresponding to the frame 40. The first correct answer data is correct answer data (that is, an annotation) for the first example data. Here, an annotation that specifies the geometric characteristics, kind, and type of the lesion included in the image used as the first example data is used as an example of the first correct answer data.

The recognition unit 82A acquires the frame 40 from the camera 52 and inputs the acquired frame 40 to the recognition model 92. Therefore, each time the frame 40 is input, the recognition model 92 specifies the geometric characteristics of the lesion 42 included in the input frame 40 and outputs information that can specify the geometric characteristics. In the example illustrated in FIG. 5, position specification information 98 that can specify the position of the lesion 42 in the frame 40 is illustrated as an example of the information that can specify the geometric characteristics.

The recognition unit 82A acquires a lesion position map 100 from the recognition model 92 and outputs the lesion position map 100. The geometric characteristics (for example, the shape and size of an outer contour) of the lesion position map 100 correspond to the geometric characteristics (for example, the shape and size of the outer contour) of the frame 40. The lesion position map 100 includes a bounding box 102. The bounding box 102 is a rectangular frame (for example, a rectangular frame circumscribing an image region indicating the lesion 42) that can specify the position recognized by the recognition model 92 as the position of the lesion 42, which is included in the frame 40, in the frame 40. The position specification information 98 is given to a geometric center 102A of the bounding box 102 (for example, a centroid of the bounding box 102). In this embodiment, the position specification information 98 is information (for example, coordinates) that can specify the position of the geometric center 102A in the frame 40. In this embodiment, the position of the geometric center 102A in the frame 40 is an example of a “recognition result” and “geometric characteristics” according to the technology of the present disclosure.

In addition, the lesion position map 100 may be displayed as the medical information 44 on the screen 35 (for example, the second display region 38) by the control unit 82C. In this case, the lesion position map 100 displayed on the screen 35 is updated according to the display frame rate applied to the first display region 36. That is, the display of the lesion position map 100 in the second display region 38 (that is, the display of the bounding box 102) is updated in synchronization with the display timing of the endoscope video image 39 in the first display region 36. This configuration enables the doctor 12 to ascertain the schematic position of the lesion 42 in the endoscope video image 39 displayed in the first display region 36 with reference to the lesion position map 100 displayed in the second display region 38 while observing the endoscope video image 39 displayed in the first display region 36.

Further, the recognition unit 82A acquires information indicating the kind and type of the lesion 42 included in the frame 40 input to the recognition model 92 from the recognition model 92 and outputs the information. The information indicating the kind and type of the lesion 42 may also be displayed as the medical information 44 on the screen 35 (for example, in the second display region 38) by the control unit 82C.

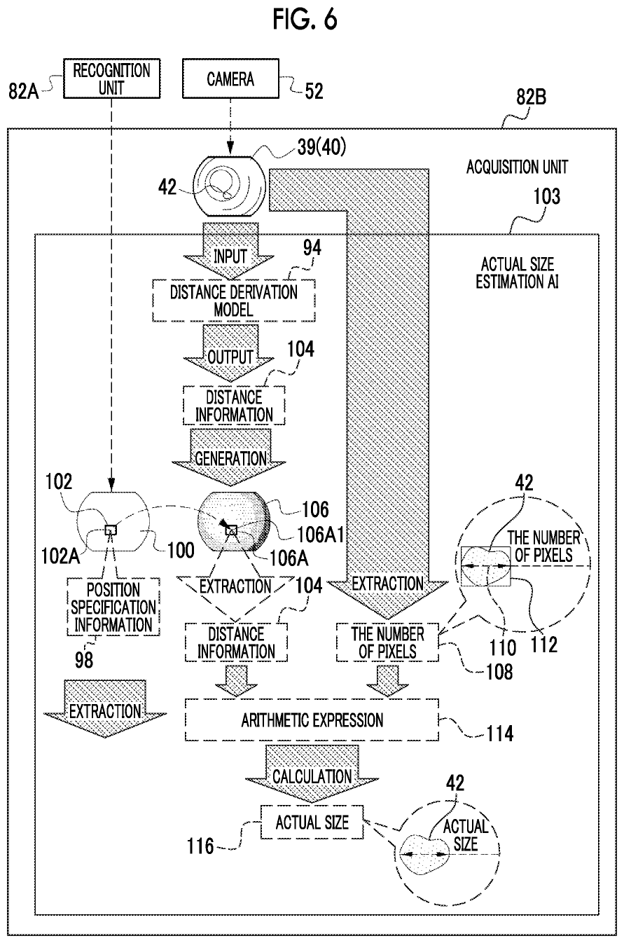

For example, as illustrated in FIG. 6, the acquisition unit 82B acquires the frame 40 from the camera 52 and acquires an actual size 116 of the lesion 42 included in the frame 40 (here, for example, the frame 40 used for the recognition process 96) acquired from the camera 52. In this embodiment, the actual size 116 is an example of an “actual size” according to the technology of the present disclosure.

The acquisition unit 82B has an actual size estimation AI 103. In this embodiment, an algorithm having AI (here, for example, the distance derivation model 94) is used as an example of the actual size estimation AI 103. The actual size estimation AI 103 estimates the actual size 116 of the lesion 42 recognized by performing the recognition process 96 (see FIG. 5) on the frame 40 including the lesion 42. In this embodiment, the actual size estimation AI 103 is an example of an “actual size estimation AI” according to the technology of the present disclosure.

The acquisition unit 82B operates the actual size estimation AI 103 to estimate the actual size 116 of the lesion 42 included in the frame 40 (here, for example, the frame 40 used for the recognition process 96) acquired from the camera 52 and acquires the actual size 116.

In this embodiment, the operation of the actual size estimation AI 103 is synonymous with the execution of the estimation of the actual size 116 using the actual size estimation AI 103 (that is, the execution of calculation using the actual size estimation AI 103 by hardware resources). The estimation of the actual size 116 using the actual size estimation AI 103 is executed by inputting the frame 40 to the actual size estimation AI 103. In this embodiment, for example, with the input of the frame 40 to the actual size estimation AI 103, the actual size estimation AI 103 operates. In a case where the frame 40 is not input to the actual size estimation AI 103 at a predetermined time interval (for example, a time interval determined according to a predetermined frame rate), the actual size estimation AI 103 does not operate (that is, stops). The fact that the actual size estimation AI 103 does not operate means that the calculation using the actual size estimation AI 103 is not performed.

The acquisition unit 82B operates the actual size estimation AI 103 on the basis of each of the plurality of frames 40 included in the endoscope video image 39 acquired from the camera 52 to acquire the actual size 116 of the lesion 42 in time series.

The acquisition unit 82B acquires distance information 104 of the lesion 42 on the basis of the frame 40 acquired from the camera 52 in order to acquire the actual size 116 of the lesion 42. The distance information 104 is information indicating a distance from the camera 52 (that is, the observation position) to the intestinal wall 32 (see FIG. 1) including the lesion 42. In addition, here, the distance from the camera 52 to the intestinal wall 32 including the lesion 42 is given as an example. However, this is only an example. Instead of the distance, a numerical value indicating a depth from the camera 52 to the intestinal wall 32 including the lesion 42 (for example, a plurality of numerical values where the depth is defined in stages (for example, numerical values in several stages to several tens of stages)) may be used.

The distance information 104 is acquired for each of all pixels constituting the frame 40. The distance information 104 may be acquired for each block (for example, each pixel group composed of several pixels to several hundreds of pixels) larger than the pixel in the frame 40.

The acquisition of the distance information 104 by the acquisition unit 82B is implemented, for example, by deriving the distance information 104 using the AI method. In this embodiment, the distance derivation model 94 is used to derive the distance information 104.

The distance derivation model 94 is optimized by performing machine learning on the neural network using second training data. The second training data is a data set including a plurality of data items (that is, data corresponding to a plurality of frames) in which second example data and second correct answer data have been associated with each other.

The second example data is an image corresponding to the frame 40. The second correct answer data is correct answer data (that is, an annotation) for the second example data. Here, an annotation that specifies the distance corresponding to each pixel included in the image used as the second example data is used as an example of the second correct answer data.

The acquisition unit 82B acquires the frame 40 from the camera 52 and inputs the acquired frame 40 to the distance derivation model 94. Then, the distance derivation model 94 outputs the distance information 104 for each pixel of the input frame 40. That is, in the acquisition unit 82B, the distance derivation model 94 outputs information indicating the distance from the position of the camera 52 (for example, the position of an image sensor or an objective lens mounted on the camera 52) to the intestinal wall 32 included in the frame 40 as the distance information 104 for each pixel of the frame 40.

The acquisition unit 82B generates a distance image 106 on the basis of the distance information 104 output from the distance derivation model 94. The distance image 106 is an image in which the distance information 104 is distributed for each pixel included in the endoscope video image 39.

The acquisition unit 82B acquires the position specification information 98 that has been given to the geometric center 102A in the lesion position map 100 obtained by the recognition unit 82A. The acquisition unit 82B extracts the distance information 104 from a bounding box correspondence region 106A in the distance image 106 with reference to the position specification information 98. The bounding box correspondence region 106A is a region corresponding to the position of the bounding box 102 in the lesion position map 100 in the entire region of the distance image 106. An example of the distance information 104 extracted from the bounding box correspondence region 106A is distance information 104 of a position 106A1 (that is, a geometric center of the bounding box correspondence region 106A) corresponding to the position (that is, the geometric center 102A) specified from the position specification information 98.

In addition, another example of the distance information 104 extracted from the bounding box correspondence region 106A is statistics (for example, a median, a mean, or a mode) of the distance information 104 for a plurality of distance information items 104 included in the bounding box correspondence region 106A (for example, a plurality of representative distance information items 104 included in the bounding box correspondence region 106A or all of the distance information items 104 included in the bounding box correspondence region 106A).

The acquisition unit 82B extracts the number of pixels 108 from the frame 40. The number of pixels 108 is the number of pixels on a line segment 110 that crosses an image region (that is, an image region indicating the lesion 42) at the position specified from the position specification information 98 in the entire image region of the frame 40 input to the distance derivation model 94. An example of the line segment 110 is the longest line segment parallel to a long side of a rectangular frame 112 that circumscribes the image region indicating the lesion 42. In addition, the line segment 110 is only an example. Instead of the line segment 110, the longest line segment parallel to a short side of the rectangular frame 112 that circumscribes the image region indicating the lesion 42 may be applied.

The acquisition unit 82B calculates the actual size 116 of the lesion 42 on the basis of the distance information 104 extracted from the bounding box correspondence region 106A in the distance image 106 and the number of pixels 108 extracted from the frame 40. An arithmetic expression 114 is used to calculate the actual size 116. The arithmetic expression 114 is a calculation expression that has the distance information 104 and the number of pixels 108 as independent variables and the actual size 116 as a dependent variable. The acquisition unit 82B inputs the distance information 104 extracted from the distance image 106 and the number of pixels 108 extracted from the frame 40 to the arithmetic expression 114. The arithmetic expression 114 outputs the actual size 116 corresponding to the input distance information 104 and the input number of pixels 108.

In addition, here, the length of the lesion 42 in the real space is given as an example of the actual size 116. However, the technology of the present disclosure is not limited thereto. The actual size 116 may be the surface area or volume of the lesion 42 in the real space. In this case, for example, an arithmetic expression that has the number of pixels of the entire image region indicating the lesion 42 and the distance information 104 as independent variables and the surface area or volume of the lesion 42 in the real space as a dependent variable is used as the arithmetic expression 114.

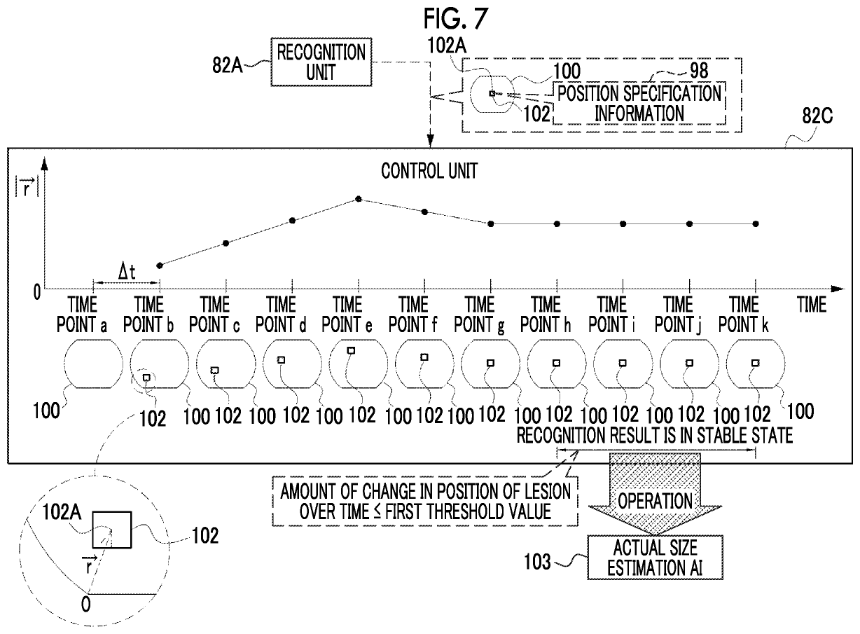

For example, as illustrated in FIG. 7, the control unit 82C acquires the position specification information 98 from the recognition unit 82A each time the recognition unit 82A performs the recognition process 96. The acquisition of the position specification information 98 from the recognition unit 82A is synonymous with the acquisition of a recognition result obtained by the recognition process 96. In addition, the control unit 82C acquires the lesion position map 100 from the recognition unit 82A.

The control unit 82C calculates a vector r on the basis of the position specification information 98 and the lesion position map 100 each time the position specification information 98 and the lesion position map 100 are acquired from the recognition unit 82A. The vector r is a vector that has the origin O set in the lesion position map 100 as a start point and the geometric center 102A specified from the position specification information 98 as an end point. The control unit 82C calculates the amount of change in the position of the lesion 42 in the frame 40 over time (hereinafter, also referred to as “an amount of change in a position of a lesion over time”) at each time interval Δt (for example, a time interval determined according to a predetermined frame rate). Here, the absolute value of the vector r (hereinafter, also referred to as a “vector absolute value”) is calculated as the amount of change in the position of the lesion over time. The amount of change in the vector absolute value over time indicates, for example, the amount of change in the vector absolute value over time obtained between the lesion position maps 100 adjacent to each other on a time axis.

The control unit 82C determines whether or not the amount of change in the position of the lesion over time is equal to or less than a first threshold value. An example of the first threshold value is a value that is defined as the maximum value of the amount of change in the position of the lesion over time at which it can be determined that the position of the lesion 42 in the frame 40 is stable. The first threshold value may be a fixed value predetermined by, for example, a computer simulation and/or a test using an actual device or may be a variable value that is changed depending on a given instruction and/or various conditions.

In a case where the amount of change in the position of the lesion over time exceeds the first threshold value, the control unit 82C determines that the recognition result obtained by the recognition process 96 of the recognition unit 82A (hereinafter, also simply referred to as a “recognition result”) is not in a stable state. In a case where the recognition result is not in a stable state (in the example illustrated in FIG. 7, time points a to g), the control unit 82C does not operate the actual size estimation AI 103. In this case, the estimation of the actual size 116 by the actual size estimation AI 103 is not performed. The fact that the estimation of the actual size 116 is not performed means that the actual size 116 is not acquired by the acquisition unit 82B.

In a case where the amount of change in the position of the lesion over time is equal to or less than the first threshold value, the control unit 82C determines that the recognition result is in a stable state. In a case where the recognition result is in a stable state (in the example illustrated in FIG. 7, time points h to k), the control unit 82C operates the actual size estimation AI 103. In a case where the actual size estimation AI 103 is operated, the actual size 116 is estimated by the actual size estimation AI 103. Then, the acquisition unit 82B acquires the actual size 116.

As described above, in the example illustrated in FIG. 7, the control unit 82C operates the actual size estimation AI 103 according to the recognition result (here, for example, the position of the lesion 42, which is included in the frame 40, in the frame 40).

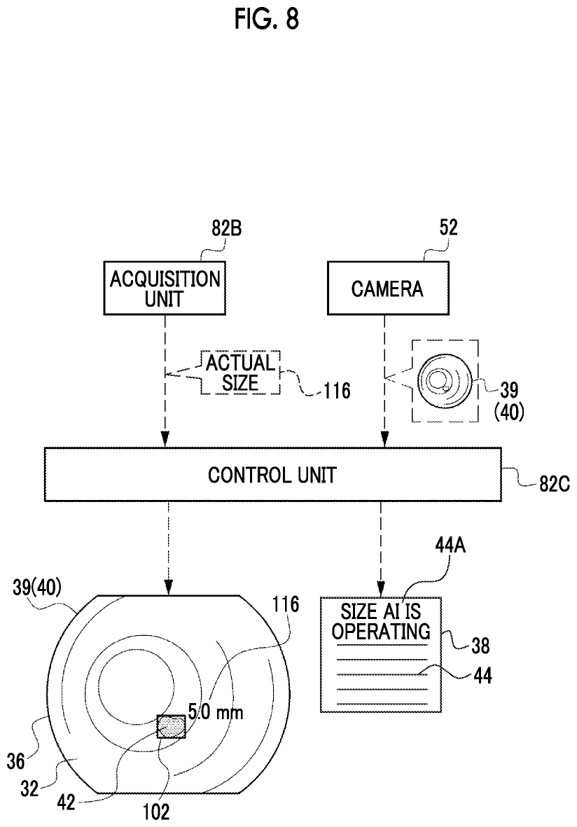

For example, as illustrated in FIG. 8, the frame 40 is displayed in the first display region 36 under the control of the control unit 82C. The control unit 82C displays the bounding box 102 in the frame 40. For example, the bounding box 102 is displayed to be superimposed on the frame 40.

In a case where the recognition result is in a stable state, the control unit 82C displays the actual size 116 acquired by the acquisition unit 82B in the frame 40. For example, the actual size 116 is displayed to be superimposed on the frame 40.

In a case where the recognition result is not in a stable state, for example, the actual size 116 is not displayed, or the display of the actual size 116 that has already been displayed is continued. In a case where the display of the actual size 116 is continued, a visual perception level for the actual size 116 may be reduced to a level at which it is difficult to visually perceive the actual size. Examples of a method for reducing the visual perception level include a method for making the actual size 116 translucent with alpha blending and/or a method for blinking the actual size 116. Further, the actual size 116 may be displayed on the screen 35 in a display aspect in which it is possible to specify that the actual size 116 is estimated in a situation in which the recognition result is not in a stable state, without reducing the visual perception level for the actual size 116 to the level at which it is difficult to visually perceive the actual size (that is, a display aspect in which the actual size 116 can be distinguished from the actual size 116 estimated in a situation in which the recognition result is in a stable state).

Each time the acquisition unit 82B acquires the actual size 116, the control unit 82C displays the latest actual size 116 in the first display region 36. That is, the actual size 116 displayed in the first display region 36 is updated to the latest actual size 116 each time the acquisition unit 82B acquires the actual size 116. The latest actual size 116 may be displayed in the second display region 38.

The control unit 82C displays, as one of the medical information items 44, operating state information 44A that can specify an operating state of the actual size estimation AI 103 in the second display region 38. For example, in a case where the recognition result is in a stable state, the control unit 82C displays, as the operating state information 44A, information capable of specifying that the actual size estimation AI 103 is operating (in the example illustrated in FIG. 8, a text “size AI is operating”) in the second display region 38. Further, for example, in a case where the recognition result is not in a stable state, the control unit 82C displays, as the operating state information 44A, information capable of specifying that the actual size estimation AI 103 is not operating in the second display region 38. In this embodiment, the operating state information 44A is an example of “operating state information” according to the technology of the present disclosure.

The content of the operating state information 44A displayed in the second display region 38 may be displayed in the first display region 36. The content of the operating state information 44A displayed in the second display region 38 is updated according to the determination result by the control unit 82C. In addition, various types of information displayed on the screen 35 may be updated for each single frame 40 or may be updated for each group of a plurality of frames 40.

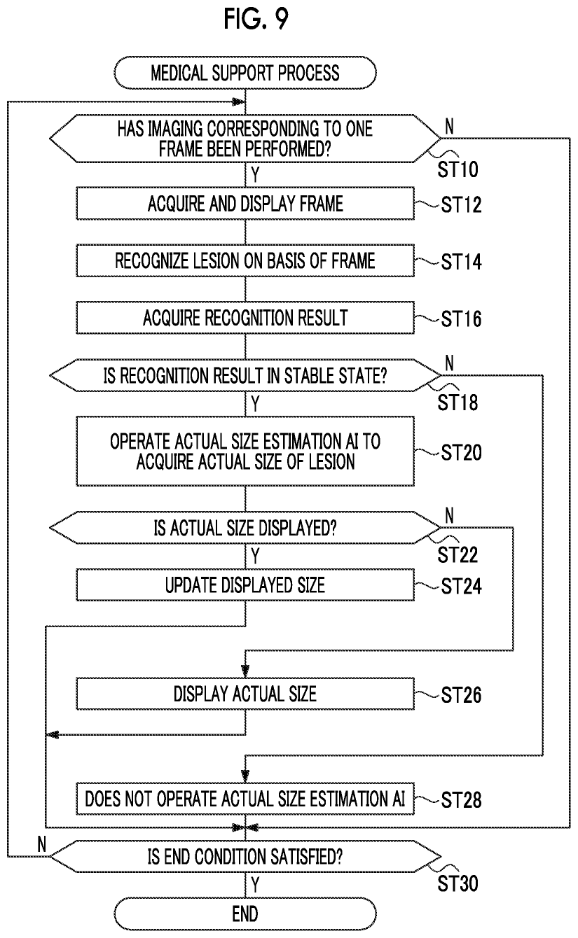

Next, the operation of a portion of the endoscope system 10 according to the technology of the present disclosure will be described with reference to FIG. 9. A flow of a medical support process illustrated in FIG. 9 is an example of the “medical support method” according to the technology of the present disclosure.

In the medical support process illustrated in FIG. 9, first, in Step ST10, the recognition unit 82A determines whether or not imaging corresponding to one frame has been performed in the large intestine 28 by the camera 52. In a case where the imaging corresponding to one frame has not been performed in the large intestine 28 by the camera 52 in Step ST10, the determination result is “No”, and the medical support process proceeds to Step ST30. In a case where the imaging corresponding to one frame has been performed in the large intestine 28 by the camera 52 in Step ST10, the determination result is “Yes”, and the medical support process proceeds to Step ST12.

In Step ST12, the recognition unit 82A and the control unit 82C acquire the frame 40 obtained by imaging the large intestine 28 with the camera 52. Then, the control unit 82C displays the frame 40 in the first display region 36 (see FIGS. 5 and 8). In addition, here, for convenience of explanation, the description will be made on the premise that the lesion 42 is included in the frame 40. After the process in Step ST12 is executed, the medical support process proceeds to Step ST14.

In Step ST14, the recognition unit 82A performs the recognition process 96 on the frame 40 acquired in Step ST12 to recognize the lesion 42 included in the frame 40 (see FIG. 5). After the process in Step ST14 is executed, the medical support process proceeds to Step ST16.

In Step ST16, the control unit 82C acquires the recognition result (here, for example, the position of the lesion 42, which is included in the frame 40, in the frame 40) obtained by the recognition process 96 in Step ST14 (see FIG. 7). The acquisition of the recognition result is implemented by acquiring the position specification information 98. After the process in Step ST16 is executed, the medical support process proceeds to Step ST18.

In Step ST18, the control unit 82C determines whether or not the recognition result is in a stable state, on the basis of the recognition result acquired in Step ST16. In a case where the recognition result is not in a stable state in Step ST18, the determination result is “No”, and the medical support process proceeds to Step ST28. In a case where the recognition result is in a stable state in Step ST18, the determination result is “Yes”, and the medical support process proceeds to Step ST20.

In Step ST20, the control unit 82C operates the actual size estimation AI 103. Therefore, the actual size estimation AI 103 estimates the actual size 116 of the lesion 42 included in the frame 40 acquired in Step ST12. The acquisition unit 82B acquires the actual size 116 estimated by the actual size estimation AI 103. After the process in Step ST20 is executed, the medical support process proceeds to Step ST22.

In Step ST22, the control unit 82C determines whether or not the actual size 116 is displayed in the frame 40. In a case where the actual size 116 is not displayed in the frame 40 in Step ST22, the determination result is “No”, and the medical support process proceeds to Step ST26. In a case where the actual size 116 is displayed in the frame 40 in Step ST22, the determination result is “Yes”, and the medical support process proceeds to Step ST24.

In Step ST24, the control unit 82C updates the actual size 116 displayed in the frame 40 to the latest actual size 116 acquired in Step ST20. After the process in Step ST24 is executed, the medical support process proceeds to Step ST30.

In Step ST26, the control unit 82C displays the actual size 116 in the frame 40. After the process in Step ST26 is executed, the medical support process proceeds to Step ST30.

In Step ST28, the control unit 82C does not operate the actual size estimation AI 103. After the process in Step ST28 is executed, the medical support process proceeds to Step ST30.

In Step ST30, the control unit 82C determines whether or not a medical support process end condition is satisfied. An example of the medical support process end condition is a condition that an instruction to end the medical support process is given to the endoscope system 10 (for example, a condition that the receiving device 64 receives the instruction to end the medical support process).

In a case where the medical support process end condition is not satisfied in Step ST30, the determination result is “No”, and the medical support process proceeds to Step ST10. In a case where the medical support process end condition is satisfied in Step ST30, the determination result is “Yes”, and the medical support process ends.

As described above, in the endoscope system 10, the actual size estimation AI 103 is operated to estimate the actual size 116 of the lesion 42 recognized by performing the recognition process 96 on the frame 40 including the lesion 42, and the acquisition unit 82B acquires the actual size 116. The control unit 82C operates the actual size estimation AI 103 according to the recognition result obtained by the recognition process 96.

For example, the control unit 82C operates the actual size estimation AI 103 in a case where the recognition result is stable and does not operate the actual size estimation AI 103 in a case where the recognition result is not stable. The actual size 116 estimated by the actual size estimation AI 103 in a case where the recognition result is stable has higher reliability than the actual size 116 estimated by the actual size estimation AI 103 in a case where the recognition result is not stable. Further, the actual size estimation AI 103 is operated in a case where the recognition result is stable. Therefore, a calculation load caused by the operation of the actual size estimation AI 103 is less than that in a case where the actual size estimation AI 103 is always operated. Therefore, according to the endoscope system 10, it is possible to achieve both highly accurate estimation of the actual size 116 of the lesion 42 and suppression of an increase in calculation load caused by the operation of the actual size estimation AI 103.

Furthermore, in the endoscope system 10, the geometric characteristics of the lesion 42 included in the frame 40 are used as the recognition result obtained by the recognition process 96. The geometric characteristics of the lesion 42 included in the frame 40 indicate, for example, the position of the lesion 42, which is included in the frame 40, in the frame 40. In this case, the control unit 82C operates the actual size estimation AI 103 according to the position of the lesion 42, which is included in the frame 40, in the frame 40. For example, the control unit 82C does not operate the actual size estimation AI 103 in a case where the position of the lesion 42, which is included in the frame 40, in the frame 40 is not stable and operates the actual size estimation AI 103 in a case where the position of the lesion 42, which is included in the frame 40, in the frame 40 is stable. Therefore, it is possible to achieve both highly accurate estimation of the actual size 116 of the lesion 42 and suppression of an increase in calculation load caused by the operation of the actual size estimation AI 103.

Moreover, in the endoscope system 10, in a case where the amount of change in the position of the lesion over time is equal to or less than the first threshold value, the control unit 82C determines that the recognition result is in a stable state. Then, the control unit 82C operates the actual size estimation AI 103 in a case where it is determined that the recognition result is in a stable state and does not operate the actual size estimation AI 103 in a case where it is determined that the recognition result is not in a stable state. Therefore, it is possible to achieve both high-accuracy estimation of the actual size 116 of the lesion 42 and suppression of an increase in calculation load caused by the operation of the actual size estimation AI 103 with high accuracy.

In addition, in the endoscope system 10, the processor 82 performs the recognition process 96 on the frame 40. Therefore, the endoscope system 10 can quickly acquire the recognition result and use the recognition result for the process using the actual size estimation AI 103.

Further, in the endoscope system 10, the processor 82 operates the actual size estimation AI 103 to estimate the actual size 116 of the lesion 42. Therefore, the endoscope system 10 can quickly acquire the actual size 116 of the lesion 42.

Furthermore, in the endoscope system 10, the actual size 116 of the lesion 42 is displayed in the first display region 36. Therefore, the doctor 12 can visually recognize the actual size 116 of the lesion 42.

Moreover, in the endoscope system 10, the operating state information 44A is displayed in the second display region 38. The operating state information 44A is information that can specify the operating state of the actual size estimation AI 103. Therefore, the doctor 12 can visually recognize the operating state of the actual size estimation AI 103.

Further, in the above-described embodiment, the form in which the control unit 82C operates the actual size estimation AI 103 in a case where the amount of change in the position of the lesion over time is equal to or less than the first threshold value is given as an example. However, the technology of the present disclosure is not limited thereto. For example, the control unit 82C may operate the actual size estimation AI 103 on the condition that the position of the lesion 42 is present in a closed region surrounding a portion of the frame 40 (hereinafter, also simply referred to as a “closed region”).

The geometric characteristics of the frame 40 correspond to the geometric characteristics of the lesion position map 100, and the closed region is set in the frame 40. Therefore, the closed region is also set in the lesion position map 100. That is, the setting content of the closed region in the frame 40 is similarly reflected in the lesion position map 100.

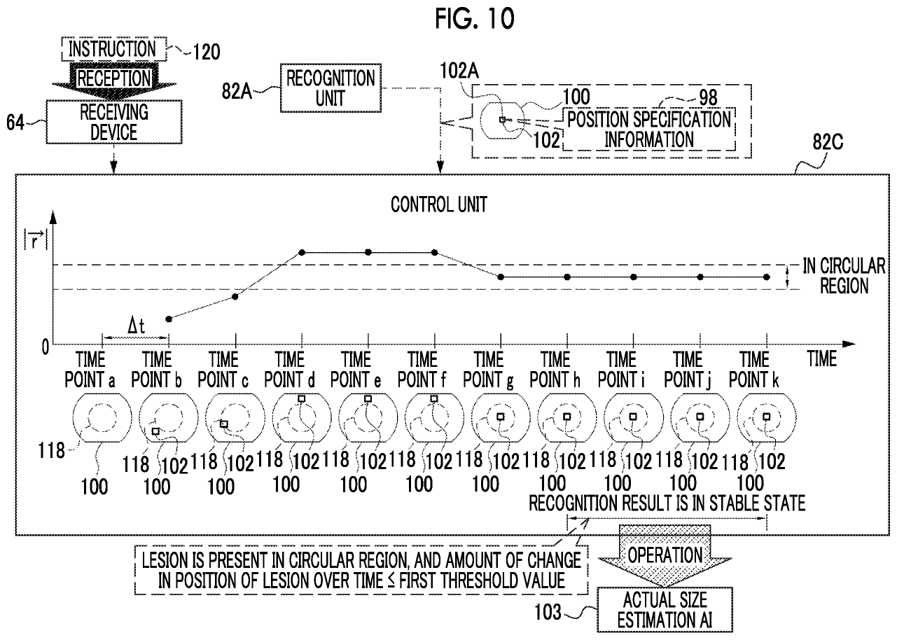

Here, in the example illustrated in FIG. 10, a circular region 118 is illustrated as the closed region. The circular region 118 is set in a central portion of the frame 40. In a case where the circular region 118 is set in the frame 40, the circular region 118 is also set in the lesion position map 100. That is, the setting content of the circular region 118 in the frame 40 is similarly reflected in the lesion position map 100. In the example illustrated in FIG. 10, the circular region 118 is disposed in a central portion of the lesion position map 100. The radius of the circular region 118 may be a fixed value or may be a variable value that is changed depending on a given instruction and/or various conditions. Since an edge portion of the frame 40 is a portion that is subjected to the optical influence of the camera 52 (for example, the influence of the aberration of a lens), it is preferable that the size of the circular region 118 is a size that does not reach the edge portion of the frame 40. In addition, here, the circular region 118 is given as an example. However, this is only an example, and a closed region having a geometric shape (for example, a polygonal shape or an elliptical shape) other than the circle may be used.

The control unit 82C determines that the recognition result is in a stable state in a case where the position of the lesion 42 is present in the circular region 118 and the amount of change in the position of the lesion over time is equal to or less than the first threshold value. In this case, the control unit 82C operates the actual size estimation AI 103. Further, the control unit 82C determines that the recognition result is not in a stable state in a case where both the condition that the position of the lesion 42 is present in the circular region 118 (hereinafter, also simply referred to as a “first condition”) and the condition that the amount of change in the position of the lesion over time is equal to or less than the first threshold value (hereinafter, also simply referred to as a “second condition”) are not established and in a case where the first condition or the second condition is not established. In this case, the control unit 82C does not operate the actual size estimation AI 103.

Therefore, for example, in a case where the circular region 118 is a region at which the doctor 12 gazes, control can be performed such that the actual size estimation AI 103 estimates the actual size 116 of the lesion 42 located in the region at which the doctor 12 gazes and does not estimate the actual size 116 of the lesion 42 located in a region other than the region at which the doctor 12 gazes. As a result, the doctor 12 can ascertain the actual size 116 of the lesion 42 located in the region at which by the doctor 12 gazes.

Further, in the example illustrated in FIG. 10, since the circular region 118 is set in the central portion of the lesion position map 100 (that is, the circular region 118 is the central portion of the frame 40), control can be performed such that the actual size estimation AI 103 estimates the actual size 116 of the lesion 42 located in the central portion of the frame 40 and does not estimate the actual size 116 of the lesion 42 located in a region other than the central portion of the frame 40. As a result, the doctor 12 can ascertain the actual size 116 of the lesion 42 located in the central portion of the frame 40.

In the example illustrated in FIG. 10, the form in which, in a case where both the first condition and the second condition are established, the control unit 82C determines that the recognition result is in a stable state and operates the actual size estimation AI 103 is given as an example. However, the technology of the present disclosure is not limited thereto. For example, in a case where only the first condition is established, the control unit 82C may determine that the recognition result is in a stable state and operate the actual size estimation AI 103.

Here, the circular region 118 is given as an example of the closed region. However, the control unit 82C may set the closed region in the frame 40 in response to a given instruction. In this case, for example, as illustrated in FIG. 10, the control unit 82C sets the closed region in the frame 40 in response to an instruction 120 received by the receiving device 64. For example, the control unit 82C sets the geometric characteristics (for example, the shape, position, and/or size) of the closed region in the frame 40 in response to the instruction 120 received by the receiving device 64. The closed region set in the frame 40 in response to the instruction 120 received by the receiving device 64 is similarly set in the lesion position map 100. In the example illustrated in FIG. 10, the circular region 118 set in the frame 40 in response to the instruction 120 received by the receiving device 64 is similarly set in the lesion position map 100. As described above, since the circular region 118 is set in the frame 40 in response to the instruction 120 received by the receiving device 64, control can be performed such that the actual size estimation AI 103 estimates the actual size 116 of the lesion 42 located in the circular region 118 intended by the doctor 12 and does not estimate the actual size 116 of the lesion 42 located in a region other than the circular region 118 intended by the doctor 12.

In the example illustrated in FIG. 10, the form in which, in a case where both the first condition and the second condition are established, it is determined that the recognition result is in a stable state and the actual size estimation AI 103 is operated is given as an example. However, the technology of the present disclosure is not limited thereto. For example, in a case where the kind of the lesion 42 is obtained as the recognition result and a condition that the kind of the lesion 42 is a predetermined kind (for example, SSL) is not established, the control unit 82C may determine that the recognition result is not in a stable state and may not operate the actual size estimation AI 103. Further, in a case where the type of the lesion 42 is obtained as the recognition result and a condition that the type of the lesion 42 is a predetermined type (for example, a superficial flat type) is not established, the control unit 82C may determine that the recognition result is not in a stable state and may not operate the actual size estimation AI 103.

In the above-described embodiment, the form in which the control unit 82C operates the actual size estimation AI 103 according to the position of the lesion 42 specified from the position specification information 98 is given as an example. However, the technology of the present disclosure is not limited thereto. For example, the control unit 82C may operate the actual size estimation AI 103 according to the approximate size of the lesion 42, which is included in the frame 40, in the frame 40 (for example, the size of the lesion 42 in a non-real space (for example, the size of the lesion 42 in the frame 40)). In this case, the same effect as that in the above-described embodiment can be expected.

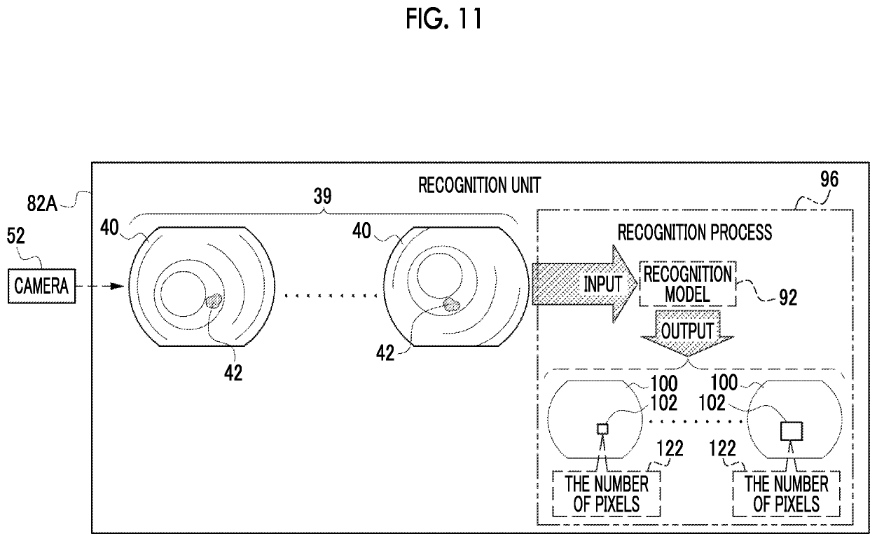

An example of the approximate size of the lesion 42, which is included in the frame 40, in the frame 40 is the size of the bounding box 102. In the example illustrated in FIG. 11, the size of the bounding box 102 is defined by the number of pixels 122 included in the bounding box 102. The number of pixels 122 is larger as the approximate size of the lesion 42, which is included in the frame 40, in the frame 40 is larger. In this embodiment, the approximate size (for example, the number of pixels 122) of the lesion 42, which is included in the frame 40, in the frame 40 is an example of a “recognition result”, “geometric characteristics”, an “approximate size”, and a “bounding box size” according to the technology of the present disclosure.

Further, here, the number of pixels 122 is given as an example of the approximate size of the lesion 42, which is included in the frame 40, in the frame 40. However, this is only an example. The approximate size of the lesion 42, which is included in the frame 40, in the frame 40 may be the area of the bounding box 102 (=“the length of the bounding box 102”×“the width of the bounding box 102”).

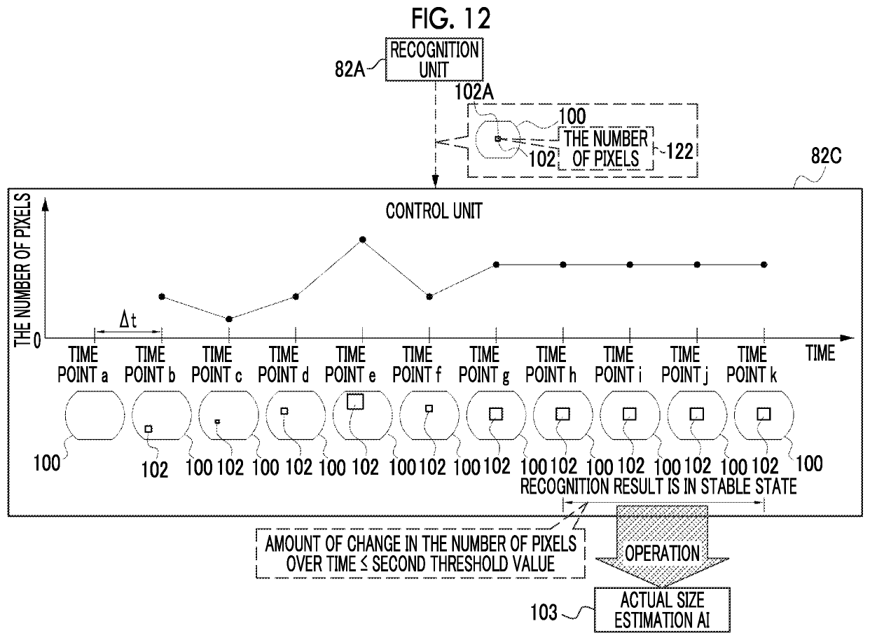

In a case where the control unit 82C operates the actual size estimation AI 103 according to the number of pixels 122, as illustrated in FIG. 12 as an example, the control unit 82C determines whether or not the amount of change in the number of pixels 122 over time is equal to or less than a second threshold value on the basis of the number of pixels 122. An example of the second threshold value is a value defined as the maximum value of the amount of change in the number of pixels 122 over time at which it can be determined that the approximate size of the lesion 42 in the frame 40, that is, the number of pixels 122 is stable. The second threshold value may be a fixed value predetermined by, for example, a computer simulation and/or a test using an actual device or may be a variable value that is changed depending on a given instruction and/or various conditions.

In a case where the amount of change in the number of pixels 122 over time exceeds the second threshold value, the control unit 82C determines that the recognition result is not in a stable state. The control unit 82C does not operate the actual size estimation AI 103 in a case where the recognition result is not in a stable state (in the example illustrated in FIG. 12, time points a to g). In this case, the estimation of the actual size 116 by the actual size estimation AI 103 is not performed. The fact that the estimation of the actual size 116 is not performed means that the actual size 116 is not acquired by the acquisition unit 82B.

In a case where the amount of change in the number of pixels 122 over time is equal to or less than the second threshold value, the control unit 82C determines that the recognition result is in a stable state. The control unit 82C operates the actual size estimation AI 103 in a case where the recognition result is in a stable state (in the example illustrated in FIG. 12, time points h to k). In a case where the actual size estimation AI 103 is operated, the actual size 116 is estimated by the actual size estimation AI 103. Then, the acquisition unit 82B acquires the actual size 116.

As described above, in the example illustrated in FIG. 12, the control unit 82C operates the actual size estimation AI 103 in a case where the amount of change in the number of pixels 122 over time is equal to or less than the second threshold value and does not operate the actual size estimation AI 103 in a case where the amount of change in the number of pixels 122 over time is greater than the second threshold value. Therefore, the same effect as that in the above-described embodiment can be expected.

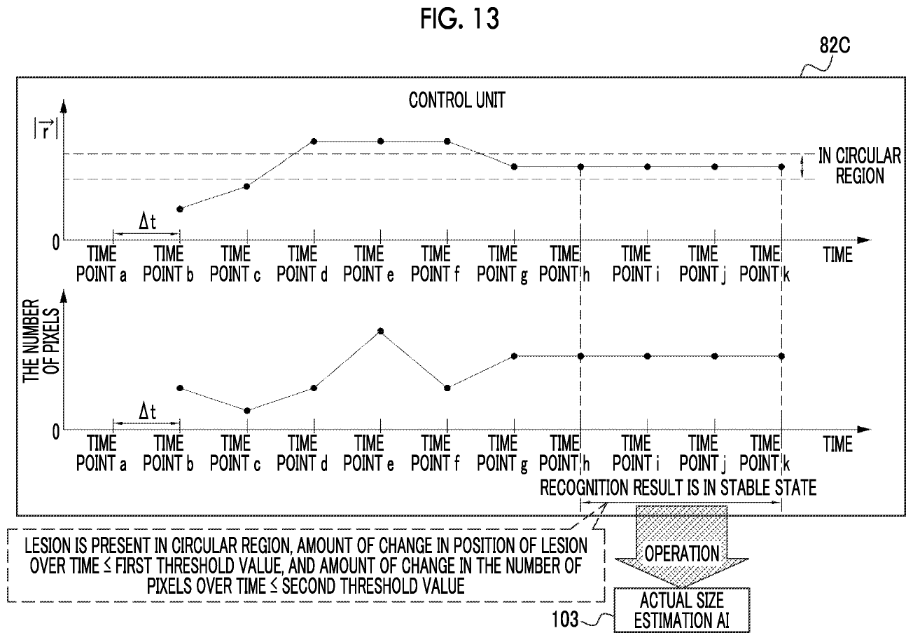

Further, for example, as illustrated in FIG. 13, in a case where both the first condition and the second condition are established and the condition that the amount of change in the number of pixels 122 over time is equal to or less than the second threshold value (hereinafter, also simply referred to as a “third condition”) is also established, the control unit 82C may determine that the recognition result is in a stable state and operate the actual size estimation AI 103. In a case where none of the first condition, the second condition, and third condition are satisfied, the control unit 82C may determine that the recognition result is not in a stable state and may not operate the actual size estimation AI 103. In this way, it is possible to achieve both highly accurate estimation of the actual size 116 of the lesion 42 and suppression of an increase in calculation load caused by the operation of the actual size estimation AI 103 with high accuracy.

Further, in a case where the second condition and the third condition are established regardless of whether or not the first condition is established, the control unit 82C may determine that the recognition result is in a stable state and operate the actual size estimation AI 103. In a case where the second condition and/or the third condition is not established, the control unit 82C may determine that the recognition result is not in a stable state and may not operate the actual size estimation AI 103. Furthermore, in a case where the second condition or the third condition is established regardless of whether or not the first condition is established, the control unit 82C may determine that the recognition result is in a stable state and operate the actual size estimation AI 103. In a case where the second condition and the third condition are not established, the control unit 82C may determine that the recognition result is not in a stable state and may not operate the actual size estimation AI 103.

In the example illustrated in FIG. 13, the form in which it is determined whether or not the amount of change in the number of pixels 122 over time is equal to or less than the second threshold value is given as an example. However, the technology of the present disclosure is not limited thereto. For example, it may be determined whether or not the amount of change in the number of pixels 122 over time is within a predetermined number-of-pixels range. An example of the predetermined number-of-pixels range is a range defined by the upper limit and lower limit of the number of pixels 122 in which it can be determined that the number of pixels 122 is stable. The predetermined number-of-pixels range may be a fixed value predetermined by a computer simulation and/or a test using an actual device or may be a variable value that is changed depending on a given instruction and/or various conditions.

In the example illustrated in FIG. 13, the form in which it is determined whether or not the amount of change in the number of pixels 122 over time is equal to or less than the second threshold value is given as an example. However, the technology of the present disclosure is not limited thereto. It may be determined whether or not the number of pixels 122 corresponding to one frame is within a reference number-of-pixels range. The reference number-of-pixels range may be set for the frame 40 in response to a given instruction in the same manner as the circular region 118 is set for the frame 40.

In the above-described embodiment, the description has been made on the premise that the recognition result is obtained for each frame 40. However, the recognition result is not obtained for each frame 40. Therefore, in a case where there is a time period in which the recognition result is missing, the recognition result in the time period in which the recognition result is missing may be interpolated and used.

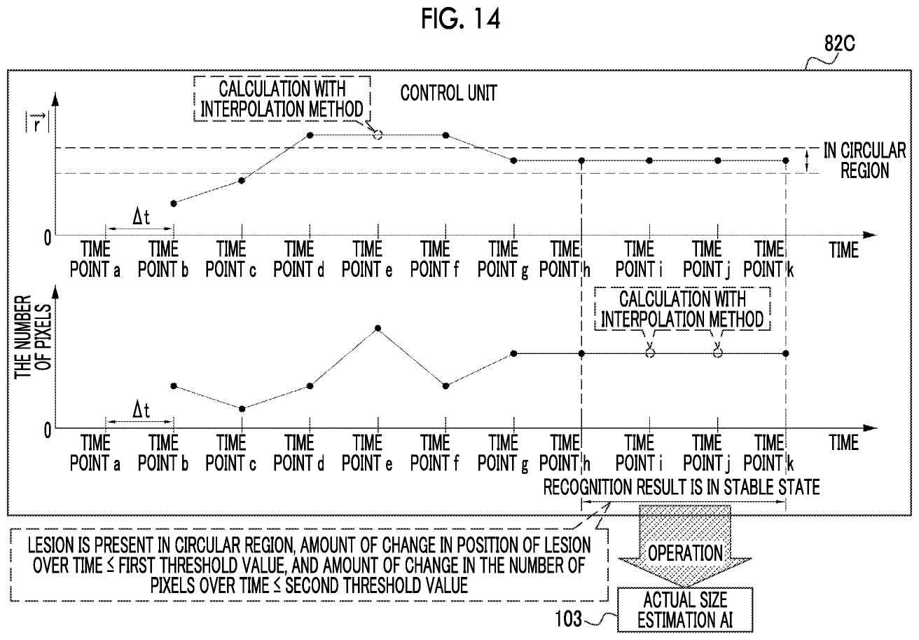

For example, as illustrated in FIG. 14, the vector absolute value in a time period (for example, a time point e) in which the vector absolute value is missing may be interpolated by an interpolation method (for example, interpolation, extrapolation, or spline interpolation) using the vector absolute value obtained on the basis of the recognition result (for example, the position of the lesion 42, which is included in the frame 40, in the frame 40) obtained before and/or after (for example, a time point d and/or a time point f) the time period in which the vector absolute value is missing. Here, an example in which the vector absolute value is calculated by the interpolation method has been described. However, the technology of the present disclosure is not limited thereto. The recognition result (for example, the position of the lesion 42, which is included in the frame 40, in the frame 40) may be calculated by the interpolation method, and the vector absolute value may be calculated using the calculated recognition result in the same manner as that in the above-described embodiment.

In addition, in a case where there is a time period in which the number of pixels 122 is missing, the number of pixels 122 in the time period in which the number of pixels 122 is missing may be interpolated and used. For example, as illustrated in FIG. 14, the number of pixels 122 in the time period (for example, time points i and j) in which the number of pixels 122 is missing may be interpolated by the interpolation method (for example, interpolation, extrapolation, or spline interpolation) using the recognition result (for example, the number of pixels 122 of the lesion 42, which is included in the frame 40, in the frame 40) obtained before and/or after (for example, a time point h and/or a time point k) the time period in which the number of pixels 122 is missing.

As described above, in a case where there is a time period in which the recognition result is not obtained, the recognition result in the time period in which the recognition result is not obtained is interpolated on the basis of the recognition result obtained before and/or after the time period in which the recognition result is not obtained, and the interpolated recognition result is used. Therefore, even in a case where there is a time period in which the recognition result is not obtained, the same effect as that in the above-described embodiment can be obtained.

In addition, the same applies to a case in which the kind of the lesion 42 and/or the type of the lesion 42 is obtained as the recognition result and, in addition to the position of the lesion 42 and/or the approximate size of the lesion 42 (for example, the number of pixels 122), the kind of the lesion 42 and/or the type of the lesion 42 is used to determine whether or not the recognition result is in a stable state. That is, in this case, the kind of the lesion 42 and/or the type of the lesion 42 in the time period in which the kind of the lesion 42 and/or the type of the lesion 42 is not obtained may be interpolated on the basis of the recognition result (here, for example, the kind of the lesion 42 and/or the type of the lesion 42) obtained before and/or after the time period in which the kind of the lesion 42 and/or the type of the lesion 42 is not obtained.

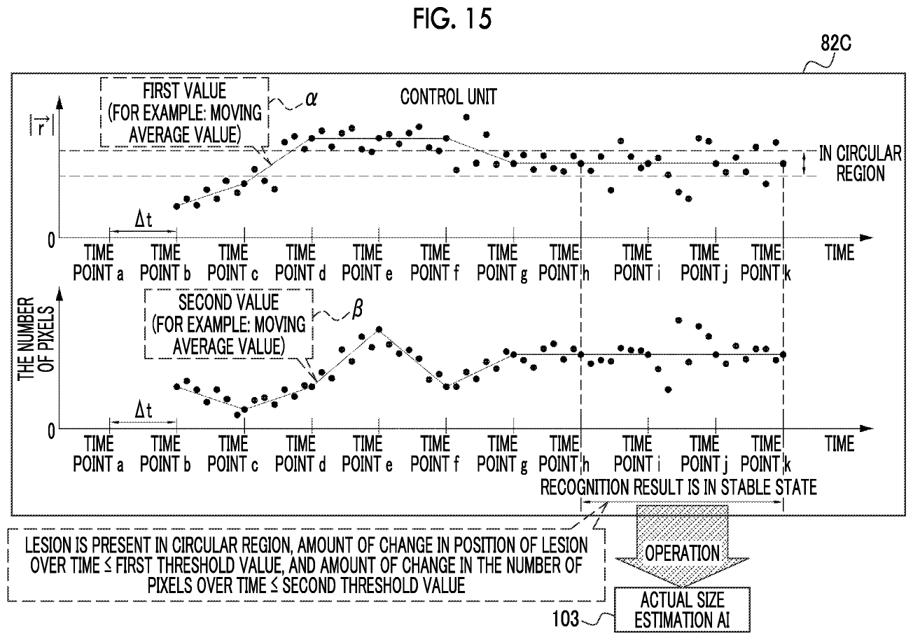

In the above-described embodiment, the form in which whether or not the recognition result is in a stable state is determined according to the vector absolute value and the number of pixels 122 obtained on the basis of the recognition results obtained at each time interval Δt has been described as an example. However, for example, even in a case where the recognition result is obtained at a time interval (hereinafter, also referred to as a “short time interval”) that is shorter than the time interval Δt by increasing the imaging frame rate, the technology of the present disclosure is established. In this case, for example, in a case where a plurality of recognition results obtained at the short time interval vary, with the variation in the recognition results, the vector absolute value and the number of pixels 122 also vary as illustrated in FIG. 15 as an example. In this case, the time period in which the recognition result is in a stable state and the time period in which the recognition result is not in a stable state are frequently switched. With the frequent switching between the time periods, a state in which the actual size estimation AI 103 is operating and a state in which the actual size estimation AI 103 is not operating are frequently switched. As a result, the display and non-display of the actual size 116 on the screen 35 are also frequently switched, which may give visual discomfort to the doctor 12 who checks the actual size 116 through the screen 35.

Therefore, the control unit 82C determines whether or not a first value α obtained by smoothing the amounts of change in a plurality of vector absolute values, which have been obtained at each short time interval, over time is equal to or less than the first threshold value and operates the actual size estimation AI 103 on the basis of the determination result. An example of the first value α is a moving average value of the amounts of change in the plurality of vector absolute values, which have been obtained at each short time interval, over time. In addition, the moving average value of the amounts of change in the plurality of vector absolute values, which have been obtained at each short time interval, over time is only an example. The first value α may be any value obtained by smoothing the amounts of change in the plurality of vector absolute values, which have been obtained at each short time interval, over time.

Further, the control unit 82C determines whether or not a second value β obtained by smoothing the amounts of change in a plurality of numbers of pixels 122, which have been obtained at each short time interval, over time is equal to or less than the second threshold value and operates the actual size estimation AI 103 on the basis of the determination result. An example of the second value β is a moving average value of the amounts of change in the plurality of numbers of pixels 122, which have been obtained at each short time interval, over time. In addition, the moving average value of the amounts of change in the plurality of numbers of pixels 122, which have been obtained at each short time interval, over time is only an example. The second value β may be any value obtained by smoothing the amounts of change in the plurality of numbers of pixels 122, which have been obtained at each short time interval, over time.

As described above, in the example illustrated in FIG. 15, it is determined whether or not the first value α is equal to or less than the first threshold value, and it is determined whether or not the second value β is equal to or less than the second threshold value. The actual size estimation AI 103 operates on the basis of the determination results. Therefore, it is possible to suppress the behavior in which the actual size estimation AI 103 suddenly operates or does not suddenly operate (that is, the behavior in which the operating state and non-operating state of the actual size estimation AI 103 are frequently switched). As a result, the frequent switching between the display and non-display of the actual size 116 on the screen 35 is suppressed. Therefore, it is possible to reduce visual discomfort given to the doctor 12 who checks the actual size 116 through the screen 35.

In the example illustrated in FIG. 15, the first value α is an example of a “first value” according to the technology of the present disclosure, and the second value β is an example of a “second value” according to the technology of the present disclosure.

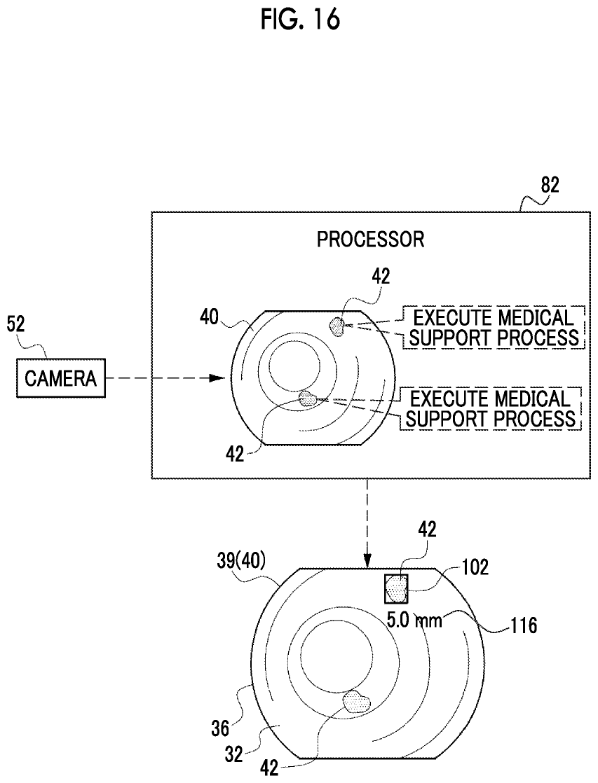

In the above-described embodiment, the form in which the medical support process is performed on the basis of the frame 40 in which one lesion 42 is included is given as an example. However, the technology of the present disclosure is not limited thereto. For example, as illustrated in FIG. 16, the technology of the present disclosure is established even in a case where a plurality of lesions 42 are included in the frame 40. In this case, the medical support process may be executed on each of the plurality of lesions 42 included in the frame 40 in the same manner as that in the above-described embodiment. Then, the bounding box 102 may be displayed for the lesion 42 corresponding to the actual size 116 displayed in the first display region 36 such that it is possible to specify the lesion 42 subjected to the medical support process and the lesion 42 whose actual size 116 is displayed.

As described above, in a case where the medical support process is executed on each of the plurality of lesions 42 included in the frame 40, for example, each lesion 42 may be recognized by the recognition process 96, an identifier may be given to each of the recognized lesions 42, and the process by the acquisition unit 82B and the process by the control unit 82C may be performed for each identifier on the basis of the recognition results.

Further, in addition to the actual size 116, information related to the lesion 42 (information indicating the kind of the lesion 42 and/or information indicating the type of the lesion 42) may be displayed on the screen 35. In this case, a mark or the like (for example, the bounding box 102) may be given to the image region of the lesion 42 corresponding to the information displayed on the screen 35 such that which lesion 42 the information is related to can be specified.

In addition, in a case where the medical support process is executed on each of the plurality of lesions 42, a list of the results (for example, a plurality of actual sizes 116) of the medical support process obtained by executing the medical support process on each of the plurality of lesions 42 may be displayed, or the results may be selectively displayed according to the instruction received by the receiving device 64 and/or various conditions. In this case, for example, information that can specify which lesion 42 the result of the medical support process corresponds to (for example, information in which the result of the medical support process and the corresponding lesion 42 are linked to be visually specified) is displayed on the screen 35.

Further, even in a case where the medical support process is executed on each of the plurality of lesions 42 included in the frame 40 in the same manner as that in the above-described embodiment, the operating state information 44A may be displayed on the screen 35 (for example, in the second display region 38) as in the above-described embodiment.

As described above, the medical support process is executed on each of the plurality of lesions 42 included in the frame 40 in the same manner as that in the above-described embodiment, which makes it possible to obtain the same effect as that in the above-described embodiment even in a case where a plurality of lesions 42 are included in the frame 40.

In the above-described embodiment, the form in which the control unit 82C generates the distance image 106 (see FIG. 6) from the frame 40 using the distance derivation model 94 (see FIG. 6) is given as an example. However, the technology of the present disclosure is not limited thereto. For example, the depth of the large intestine 28 in a depth direction may be measured by a depth sensor (for example, a sensor that measures a distance using a laser distance measurement method and/or a phase difference method) provided in the distal end part 50 (see FIG. 2), and the processor 82 may generate the distance image 106 on the basis of the measured depth.

In the above-described embodiment, the case where the recognition process 96 is performed by the bounding box method using AI has been described. However, this is only an example. An object recognition process (for example, semantic segmentation, instance segmentation, and/or panoptic segmentation) may be performed by a segmentation method using AI.

In this case, the recognition model 92 is a trained model for object recognition in the segmentation method using AI. An example of the trained model for object recognition in the segmentation method using AI is a model for semantic segmentation. An example of the model for semantic segmentation is a model having an encoder-decoder structure. An example of the model having an encoder-decoder structure is U-Net or HRNet.

Further, in a case where the recognition process 96 is implemented by the object recognition process in the segmentation method using AI, a probability map may be used instead of the lesion position map 100. The probability map is a map in which a distribution of the position of the lesion 42 in the frame 40 is represented by a probability which is an example of an indicator indicating likelihood. Furthermore, in general, the probability map is also referred to as a reliability degree map, a certainty degree map, or the like.

The probability map includes a segmentation image that defines the lesion 42 recognized by the recognition unit 82A. The segmentation image is an image region that specifies the position of the lesion 42 in the frame 40 recognized by performing the recognition process 96 on the frame 40 (that is, an image displayed in a display aspect that can specify the position where the lesion 42 is most likely to be present in the frame 40). Information that can specify the position of the segmentation image in the frame 40 (for example, information (for example, coordinates) that can specify the position of the centroid of the segmentation image in the frame 40) is associated as the position specification information 98 with the segmentation image by the recognition unit 82A.

In a case where the recognition process 96 is implemented by the object recognition process in the segmentation method using AI, the segmentation image is used instead of the bounding box 102. In this case, for example, information that can specify the position of the segmentation image in the frame 40 may be used as the position specification information 98, and the number of pixels of the segmentation image may be used as the number of pixels 122.

In the above-described embodiment, the form in which the length of the longest range crossing the lesion 42 along the line segment 110 in the real space is measured as the actual size 116 is given as an example. However, the technology of the present disclosure is not limited thereto. For example, the length of a range, which corresponds to the longest line segment parallel to the short side of the rectangular frame 112 for the image region indicating the lesion 42, in the real space may be measured as the actual size 116 and displayed on the screen 35. In this case, the doctor 12 can ascertain the length of the longest range, which crosses the lesion 42 along the longest line segment parallel to the short side of the rectangular frame 112 for the image region indicating the lesion 42, in the real space.

In addition, the size of the lesion 42 with respect to the radius and/or diameter of a circle that circumscribes the image region indicating the lesion 42 in the real space may be measured and displayed on the screen 35. In this case, the doctor 12 can ascertain the size of the lesion 42 with respect to the radius and/or diameter of the circle that circumscribes the image region indicating the lesion 42 in the real space.

In the above-described embodiment, the form in which the actual size 116 is displayed in the first display region 36 is given as an example. However, this is only an example. The actual size 116 may be displayed in a pop-up manner from the inside of the first display region 36 to the outside of the first display region 36, or the actual size 116 may be displayed in a region other than the first display region 36 in the screen 35. In addition, for example, the kind of lesion and/or the type of lesion may also be displayed in the first display region 36 and/or the second display region 38 or may be displayed on a screen other than the screen 35.

In the above-described embodiment, the form in which whether or not the recognition result is in a stable state is determined on the basis of the amount of change in the position of the lesion over time between two frames adjacent to each other in terms of time has been described as an example. However, the technology of the present disclosure is not limited thereto. Whether or not the recognition result is in a stable state may be determined on the basis of the amount of change in the position of the lesion over time among three or more frames adjacent to each other in terms of time. The same applies to the amount of change in the number of pixels 122 over time.

In the above-described embodiment, the form in which the estimation of the actual size 116 is performed for each frame is given as an example. However, this is only an example. The estimation of the actual size 116 may be performed for each group of a plurality of frames. In addition, a representative size (for example, a mean, a median, a maximum value, a minimum value, a deviation, a standard deviation, and/or a mode) obtained by performing the estimation of the actual size 116 for each group of a plurality of frames may be displayed on the screen 35.

In the above-described embodiment, the object recognition process using the AI method is given as an example of as the recognition process 96. However, the technology of the present disclosure is not limited thereto. An object recognition process using a non-AI method (for example, template matching) may be executed such that the recognition unit 82A recognizes the lesion 42 included in the frame 40.

In the above-described embodiment, the form in which the distance information 104 extracted from the bounding box correspondence region 106A in the distance image 106 is input to the arithmetic expression 114 is given as an example. However, the technology of the present disclosure is not limited thereto. For example, the distance image 106 may not be generated, the distance information 104 corresponding to the position specified from the position specification information 98 may be extracted from all of the distance information items 104 output from the distance derivation model 94, and the extracted distance information 104 may be input to the arithmetic expression 114.

In the above-described embodiment, the form in which the distance information 104 is derived using the distance derivation model 94 has been described as an example. However, the technology of the present disclosure is not limited thereto. For example, another method for deriving the distance information 104 using the AI method is a method that combines segmentation and depth estimation (for example, regression learning that gives the distance information 104 to the entire image (for example, all pixels constituting the image) or unsupervised learning that unsupervisedly learns the distance of the entire image).

In the above-described embodiment, the form in which the arithmetic expression 114 is used to calculate the actual size 116 has been described as an example. However, the technology of the present disclosure is not limited thereto. A process using AI may be performed on the frame 40 to estimate the actual size 116. In this case, for example, a trained model that outputs the actual size 116 of the lesion 42 in a case where the frame 40 including the lesion 42 is input may be used. In a case where the trained model that outputs the actual size 116 of the lesion 42 is created, deep learning may be performed on the neural network, using training data obtained by giving an annotation indicating the size of the lesion as correct answer data to the lesion included in the image used as example data, to optimize the neural network. In addition, in this case, the neural network optimized by the deep learning using the training data obtained by giving the annotation indicating the size of the lesion as the correct answer data to the lesion included in the image used as the example data is an example of “actual size estimation AI” according to the technology of the present disclosure.

In the above-described embodiment, the endoscope video image 39 is given as an example. However, the technology of the present disclosure is not limited thereto. The technology of the present disclosure is also established for medical video images (for example, video images, such as radiographic video images or ultrasound video images, obtained by a modality (for example, a radiological diagnostic apparatus or an ultrasound diagnostic apparatus) other than the endoscope system 10) other than the endoscope video image 39.

In the above-described embodiment, the form in which the actual size 116 of the lesion 42 included in the video image is estimated is given as an example. However, this is only an example. The technology of the present disclosure is also established even in a case where the actual size 116 of the lesion 42 included in frame-by-frame images or still images is estimated.

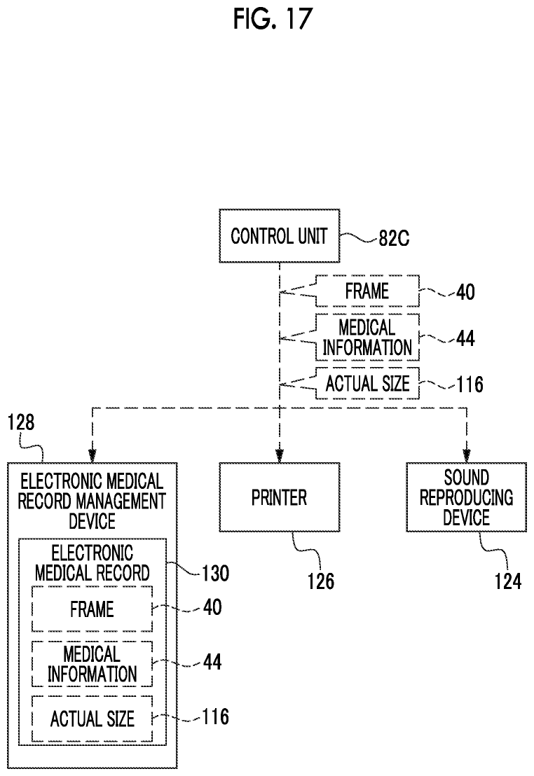

In the above-described example, the display device 18 is given as an example of the output destination of the frame 40, the medical information 44, the actual size 116, and the like. However, the technology of the present disclosure is not limited thereto. The output destination of various types of information, such as the frame 40, the medical information 44, and the actual size 116 (hereinafter, referred to as “various types of information”), may be a device other than the display device 18. For example, as illustrated in FIG. 17, a sound reproducing device 124 is given as an example of the output destination of information that can be output as sound among the various types of information. Among the various types of information, the information that can be output as sound may be output as sound by the sound reproducing device 124. In addition, examples of the output destination of the various types of information include a printer 126 and/or an electronic medical record management device 128. The various types of information may be printed as a text or the like on a medium (for example, paper) or the like by the printer 126 or may be stored in an electronic medical record 130 that is managed by the electronic medical record management device 128.

In the above-described example, the form in which various types of information are displayed on the screen 35 or various types of information are not displayed on the screen 35 has been described as an example. The display of various types of information on the screen 35 means that various types of information are displayed to be perceivable by the user (for example, the doctor 12). In addition, the concept that various types of information are not displayed on the screen 35 also includes the concept of reducing a display level of various types of information (for example, a level at which various types of information are perceived by the display). For example, the concept that various types of information are not displayed on the screen 35 also includes the concept that various types of information are displayed in a display aspect in which the various types of information are not visually perceived by the user or the like. An example of the display aspect in this case is a display aspect in which various types of information are reduced in font size, various types of information are thinned, various types of information are dotted, various types of information are blinked, various types of information are displayed at an imperceptible display time, or various types of information are displayed to be transparent to an imperceptible level. In addition, the same applies to various types of outputs such as the above-described sound output, printing, and storage.

In the above-described embodiment, the form in which the medical support process is performed by the processor 82 included in the endoscope system 10 has been described as an example. However, the technology of the present disclosure is not limited thereto. The device that performs at least some of the processes included in the medical support process may be provided outside the endoscope system 10.

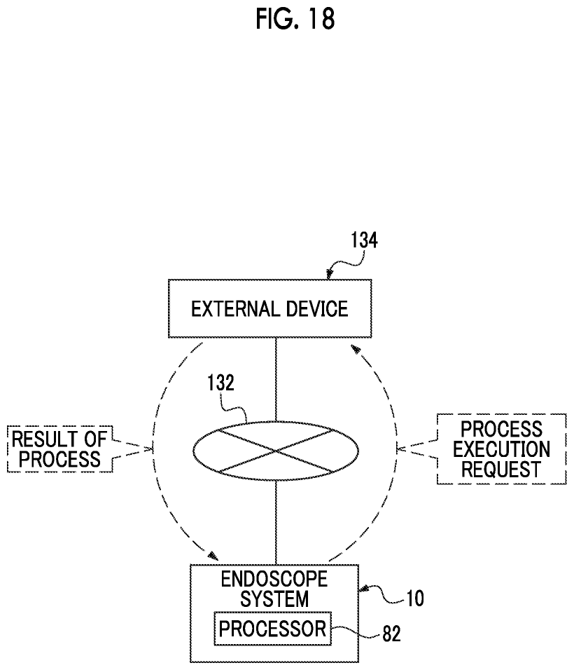

In this case, for example, as illustrated in FIG. 18, an external device 134 that is connected to the endoscope system 10 via a network 132 (for example, a WAN and/or a LAN) to communicate therewith may be used.

An example of the external device 134 is at least one server that directly or indirectly transmits and receives data to and from the endoscope system 10 via the network 132. The external device 134 receives a process execution instruction given from the processor 82 of the endoscope system 10 via the network 132. Then, the external device 134 executes a process corresponding to the received process execution instruction and transmits a result of the process to the endoscope system 10 via the network 132. In the endoscope system 10, the processor 82 receives the result of the process transmitted from the external device 134 via the network 132 and executes a process using the received result of the process.

An example of the process execution instruction is an instruction for the external device 134 to execute at least a portion of the medical support process. A first example of the at least a portion (that is, a process to be executed by the external device 134) of the medical support process is the recognition process 96. In this case, the external device 134 executes the recognition process 96 in response to the process execution instruction given from the processor 82 of the endoscope system 10 via the network 132 and transmits the result of the recognition process (for example, the position specification information 98 and/or the lesion position map 100) to the endoscope system 10 via the network 132. In the endoscope system 10, the processor 82 receives the result of the recognition process and executes the same process as that in the above-described embodiment using the received result of the recognition process.

A second example of the at least a portion (that is, the process to be executed by the external device 134) of the medical support process is the process by the actual size estimation AI 103. In this case, the external device 134 executes the process by the actual size estimation AI 103 in response to the process execution instruction given from the processor 82 of the endoscope system 10 via the network 132 and transmits the result of the measurement process (for example, the actual size 116) to the endoscope system 10 via the network 132. In the endoscope system 10, the processor 82 receives the result of the measurement process and executes the same process as that in the above-described embodiment using the received result of the measurement process.

A third example of the at least a portion (that is, the process to be executed by the external device 134) of the medical support process is the process of determining whether or not the recognition result is in a stable state and/or the process of controlling the operation of the actual size estimation AI 103.

For example, the external device 134 is implemented by cloud computing. In addition, this is only an example, and the external device 134 may be implemented by network computing, such as fog computing, edge computing, or grid computing. Instead of the server, at least one personal computer or the like may be used as the external device 134. Further, an arithmetic device with a communication function that is provided with a plurality of types of AI functions may be used.

Further, in the above-described embodiment, the form in which the medical support program 90 is stored in the NVM 86 has been described as an example. However, the technology of the present disclosure is not limited thereto. For example, the medical support program 90 may be stored in a portable non-transitory computer readable storage medium such as an SSD or a USB memory. The medical support program 90 stored in the non-transitory storage medium is installed in the computer 78 of the endoscope system 10. The processor 82 executes the medical support process according to the medical support program 90.

In addition, the medical support program 90 may be stored in a storage device of another computer, a server, or the like that is connected to the endoscope system 10 via the network. Then, the medical support program 90 may be downloaded and installed in the computer 78 in response to a request from the endoscope system 10.

In addition, the entire medical support program 90 does not need to be stored in a storage device of another computer, a server, or the like that is connected to the endoscope system 10, or the entire medical support program 90 does not need to be stored in the NVM 86. A portion of the medical support program 90 may be stored.

The following various processors can be used as hardware resources for performing the medical support process. An example of the processor is a CPU which is a general-purpose processor that executes software, that is, a program, to function as the hardware resource for performing the medical support process. In addition, an example of the processor is a dedicated electric circuit which is a processor having a dedicated circuit configuration designed to perform a specific process, such as an FPGA, a PLD, or an ASIC. Any processor has a memory provided therein or connected thereto. Any processor uses the memory to perform the medical support process.

The hardware resource for performing the medical support process may be configured by one of the various processors or by a combination of two or more processors of the same type or different types (for example, a combination of a plurality of FPGAs or a combination of a CPU and an FPGA). Further, the hardware resource for performing the medical support process may be one processor.

A first example of the configuration in which the hardware resource is configured by one processor is an aspect in which one processor is configured by a combination of one or more CPUs and software and functions as the hardware resource for performing the medical support process. A second example of the configuration is an aspect in which a processor that implements the functions of the entire system including a plurality of hardware resources for performing the medical support process using one IC chip is used. A representative example of this aspect is an SoC. As described above, the medical support process is achieved using one or more of the various processors as the hardware resource.

In addition, specifically, an electronic circuit obtained by combining circuit elements, such as semiconductor elements, can be used as the hardware structure of the various processors. Further, the above-described medical support process is only an example. Therefore, it goes without saying that unnecessary steps may be deleted, new steps may be added, or the processing order may be changed, without departing from the gist.

The content described and illustrated above is a detailed description of portions related to the technology of the present disclosure and is only an example of the technology of the present disclosure. For example, the description of the configurations, functions, operations, and effects is the description of examples of the configurations, functions, operations, and effects of the portions related to the technology of the present disclosure. Therefore, it goes without saying that unnecessary portions may be deleted or new elements may be added or replaced in the content described and illustrated above, without departing from the gist of the technology of the present disclosure. In addition, the description of, for example, common technical knowledge that does not need to be particularly described to enable the implementation of the technology of the present disclosure is omitted in the content described and illustrated above in order to avoid confusion and to facilitate the understanding of the portions related to the technology of the present disclosure.

In the specification, “A and/or B” is synonymous with “at least one of A or B”. That is, “A and/or B” means that it may be only A, only B, or a combination of A and B. Further, in the specification, the same concept as “A and/or B” is applied to a case where the connection of three or more matters is expressed by “and/or”.

All of the documents, the patent applications, and the technical standards described in the specification are incorporated by reference herein to the same extent as each individual document, each patent application, and each technical standard is specifically and individually stated to be incorporated by reference.

What is claimed is:

1. A medical support device comprising:
a processor,
wherein an actual size estimation AI is operated to estimate an actual size of a region to be observed which has been recognized by performing a recognition process on a medical image including the region to be observed, and
the processor is configured to:
acquire the actual size; and
operate the actual size estimation AI according to a recognition result obtained by the recognition process.

2. The medical support device according to claim 1,
wherein the recognition result includes geometric characteristics of the region to be observed in the medical image.

3. The medical support device according to claim 2,
wherein the geometric characteristics include a position and/or an approximate size of the region to be observed in the medical image.

4. The medical support device according to claim 3,
wherein the processor is configured to operate the actual size estimation AI according to an amount of change in the position over time and/or an amount of change in the approximate size over time.

5. The medical support device according to claim 4,
wherein the processor is configured to operate the actual size estimation AI in a case where the amount of change in the position over time is equal to or less than a first threshold value and/or in a case where the amount of change in the approximate size over time is equal to or less than a second threshold value.

6. The medical support device according to claim 4,
wherein the processor is configured to operate the actual size estimation AI in a case where a first value obtained by smoothing the amount of change in the position over time is equal to or less than a first threshold value and/or in a case where a second value obtained by smoothing the amount of change in the approximate size over time is equal to or less than a second threshold value.

7. The medical support device according to claim 6,
wherein the first value is a moving average value of the amount of change in the position over time.

8. The medical support device according to claim 6,
wherein the second value is a moving average value of the amount of change in the approximate size over time.

9. The medical support device according to claim 3,
wherein the recognition process is a process that uses an object recognition AI using a bounding box method, and
the approximate size is a bounding box size that is applied to the region to be observed by the object recognition AI.

10. The medical support device according to claim 3,
wherein the geometric characteristics include the position, and
the processor is configured to operate the actual size estimation AI on a condition that the position is present in a closed region surrounding a portion of the medical image.

11. The medical support device according to claim 10,
wherein the closed region is set in a central portion of the medical image.

12. The medical support device according to claim 10,
wherein the closed region is set in the medical image in response to a given instruction.

13. The medical support device according to claim 1,
wherein a plurality of the regions to be observed are included in the medical image,
the recognition process is a process of recognizing each of the plurality of regions to be observed which are included in the medical image,
the recognition result is obtained for each of the plurality of regions to be observed,
the actual size estimation AI is operated for each of the plurality of regions to be observed which have been recognized by performing the recognition process on the medical image, and
the processor is configured to operate the actual size estimation AI for each of the plurality of regions to be observed according to the recognition result for each of the plurality of regions to be observed.

14. The medical support device according to claim 1,
wherein, in a case where there is a time period in which the recognition result is not obtained by the recognition process, the recognition result in the time period is interpolated on the basis of the recognition result obtained by the recognition process before and/or after the time period.

15. The medical support device according to claim 1,
wherein the processor is configured to perform the recognition process on the medical image.

16. The medical support device according to claim 1,
wherein the processor is configured to operate the actual size estimation AI to estimate the actual size.

17. The medical support device according to claim 1,
wherein the processor is configured to output the actual size.

18. The medical support device according to claim 17,
wherein the output of the actual size is implemented by displaying the actual size on a screen.

19. The medical support device according to claim 1,
wherein the processor is configured to output operating state information that is capable of specifying an operating state of the actual size estimation AI.

20. The medical support device according to claim 19,
wherein the output of the operating state information is implemented by displaying the operating state information on a screen.

21. The medical support device according to claim 1,
wherein the medical image is an endoscope image captured by an endoscope.

22. The medical support device according to claim 1,
wherein the region to be observed is a lesion.

23. An endoscope system comprising:
the medical support device according to claim 1; and
an endoscope that images a region to be observed.

24. A medical support method comprising:
operating an actual size estimation AI to estimate an actual size of a region to be observed which has been recognized by performing a recognition process on a medical image including the region to be observed;
acquiring the actual size; and
operating the actual size estimation AI according to a recognition result obtained by the recognition process.

25. The medical support method according to claim 24, further comprising:
using an endoscope that images the region to be observed.

26. A non-transitory computer-readable storage medium storing a program executable by a computer to execute a medical support process comprising:
operating an actual size estimation AI to estimate an actual size of a region to be observed which has been recognized by performing a recognition process on a medical image including the region to be observed;
acquiring the actual size; and
operating the actual size estimation AI according to a recognition result obtained by the recognition process.
